(19) United States
(12) Patent Application Publication
(10) Pub. No.: US 2019/0135885 A1
(43) Pub. Date: May 9, 2019

(54) COMPOSITION COMPRISING AN AB6 FAMILY DESIGNER LIGAND OF TGF-BETA SUPERFAMILY FOR TREATING LIVER DISEASE AND USE THEREOF

(71) Applicant: MOGAM INSTITUTE FOR BIOMEDICAL RESEARCH, Yongin-si, Gyeonggi-do (KR)

(72) Inventors: Sen yon CHOE, Yongin-si (KR); Innokentiy MASLENNIKOV, Irvine, CA (US); Yong Chul KIM, Yongin-si (KR); Jung Yoon CHOI, Yongin-si (KR)

(73) Assignee: MOGAM INSTITUTE FOR BIOMEDICAL RESEARCH, Yongin-si, Gyeonggi-do (KR)

(21) Appl. No.: 16/306,058

(22) PCT Filed: May 31, 2017
(86) PCT No.: PCT/KR2017/005706

§ 371 Date: Nov. 30, 2018

Related U.S. Application Data
(60) Provisional application No. 62/343,395, filed on May 31, 2016.

(51) Int. Cl. C07K 14/495; C07K 14/51; C07K 14/575; A61P 1/16; A61K 38/00

(52) U.S. Cl. CPC C07K 14/495; C07K 14/51; A61K 38/00; A61P 1/16; C07K 14/575

(57) ABSTRACT

A composition and a method for treating a liver disease (including, but not limited to, non-alcoholic fatty liver disease, liver fibrosis and hepatic inflammation) are disclosed. The composition comprises a chimeric polypeptide having TGF-beta activity. The method includes administering an effective amount of the composition to a subject in need of treating a liver disease.

### TECHNICAL FIELD

The present invention relates to a composition comprising a chimeric polypeptide having a TGF-beta activity and a method for treating a liver disease.

### BACKGROUND ART

Activins and Bone Morphogenetic Proteins (BMPs) are members of the much larger Transforming Growth Factor-beta (TGF-β) superfamily. Due to their pervasiveness in numerous developmental and cellular processes, TGF-β ligands have been the focus of great interest.

### DISCLOSURE OF INVENTION

### Technical Problem

For TGF-β ligands to be successfully used as therapeutic tools, several hurdles need to be overcome. For example, the TGF-β ligands, which are considered as a therapeutic tool, are preferably be able to be specifically modified and altered for modulating various properties thereof. In addition, they also should be feasible for a production in a significant quantity.

### Solution to Problem

1. A composition for treating a liver disease, wherein the composition comprises a chimeric polypeptide having at least 95% sequence identity to an amino acid sequence comprising a first, a second, a third, a fourth, a fifth, and a sixth domains of amino acid residues, wherein the first domain of amino acid residues is selected from a group consisting of amino acid residues 1 to X_{1b} of SEQ ID NO:2, amino acid residues 1 to X₁ₐₐ of SEQ ID NO:4, amino acid residues 1 to X_{1ab} of SEQ ID NO:6, amino acid residues 1 to X_{1ac} of SEQ ID NO:8, and amino acid residues 1 to X₁ₐₑ of SEQ ID NO:10; the second domain of amino acid residues is selected from a group consisting of amino acid residues X_{1b} to X_{2b}, of SEQ ID NO:2, amino acid residues X₁ₐₐ to X₂ₐₐ of SEQ ID NO:4, amino acid residues X_{1ab} to X_{2ab} of SEQ ID NO:6, amino acid residues X_{1ac} to X_{2ac} of SEQ ID NO:8, and amino acid residues X₁ₐₑ to X₂ₐₑ of SEQ ID NO:10; the third domain of amino acid residues is selected from a group consisting of amino acid residues X_{2b} to X_{3b} of SEQ ID NO:2, amino acid residues X₂ₐₐ to X₃ₐₐ of SEQ ID NO:4, amino acid residues X_{2ab} to X_{3ab} of SEQ ID NO:6, amino acid residues X_{2ac} to X_{3ac} of SEQ ID NO:8, and amino acid residues X₂ₐₑ to X₃ₐₑ of SEQ ID NO:10; the fourth domain of amino acid residues is selected from a group consisting of amino acid residues X_{3b} to X_{4b} of SEQ ID NO:2, amino acid residues X₃ₐₐ to X₄ₐₐ of SEQ ID NO:4, amino acid residues X_{3ab} to X_{4ab} of SEQ ID NO:6, amino acid residues X_{3ac} to X_{4ac} of SEQ ID NO:8, and amino acid residues X₃ₐₑ, to X₄ₐₑ of SEQ ID NO:10; the fifth domain of amino acid residues is selected from a group consisting of amino acid residues X_{4b} to X_{5b} of SEQ ID NO:2, amino acid residues X₄ₐₐ to X₅ₐₐ of SEQ ID NO:4, amino acid residues X_{4ab} to X_{5ab} of SEQ ID NO:6, amino acid residues X_{4ac} to X_{5ac} of SEQ ID NO:8, and amino acid residues X₄ₐₑ to X₅ₐₑ of SEQ ID NO:10; and the sixth domain of amino acid residues is selected from a group consisting of amino acid residues X_{5b} to X_{6b} of SEQ ID NO:2, amino acid residues X₅ₐₐ to X₆ₐₐ of SEQ ID NO:4, amino acid residues X_{5ab} to X_{6ab} of SEQ ID NO:6, amino acid residues X_{5ac} to X_{6ac} of SEQ ID NO:8, and amino acid residues X₅ₐₑ to X₆ₐₑ of SEQ ID NO:10; wherein X_{1b} is 43, 44, 45, 46, 47, 48, 49, 50, 51, or 52; X_{2b} is 61, 62, 63, 64, 65, 66, 67, 68, 69, or 70; X_{3b} is 75, 76, 77, 78, 79, 80, 81, 82, 83, 84, 85, 86, 87, or 88; X_{4b} is 95, 96, 97, 98, 99, 100, 101, 102, or 103; X_{5b} is 107, 108, 109, 110, 111, 112, 113, 114, or 115; X_{6b} is 130, 131, or 132; X₁ₐₐ is 22, 23, 24, 25, 26, 27, 28, 29, 30, or 31; X₂ₐₐ is 41.42, 43, 44, 45, 46, 47, 48, 49, 50, or 51; X₃ₐₐ is 55, 51, 52, 53, 54, 55, 56, 57, 58, 59, 60, 61, 62, 63, 64, 65, 66, 67, 68, 69, 70, 71, 72, 73, or 74; X₄ₐₐ is 79, 80, 81, 82, 83, 84, 85, 86, or 87; X₅ₐₐ is 91, 92, 93, 94, 95, 96, 97, 98, 99, or 100; X₆ₐₐ is 114, 115, or 116; X_{1ab} is 22, 23, 24, 25, 26, 27, 28, 29, 30, or 31; X_{2ab} is 41, 42, 43, 44, 45, 46, 47, 48, 49, 50, or 51; X_{3ab} is 55, 51, 52, 53, 54, 55, 56, 57, 58, 59, 60, 61, 62, 63, 64, 65, 66, 67, 68, 69, 70, 71, 72, 73, or 74; X_{4ab} is 78, 79, 80, 81, 82, 83, 84, 85, or 86; X_{5ab} is 90, 91, 92, 93, 94, 95, 96, 97, 98, or 99; X_{6ab} is 113, 114, or 115; X_{1ac} is 22, 23, 24, 25, 26, 27, 28, 29, 30, or 31; X_{2ac} is 41, 42, 43, 44, 45, 46, 47, 48, 49, 50, or 51; X_{3ac} is 55, 51, 52, 53, 54, 55, 56, 57, 58, 59, 60, 61, 62, 63, 64, 65, 66, 67, 68, 69, 70, 71, 72, 73, or 74; X_{4ac} is 79, 80, 81, 82, 83, 84, 85, 86, or 87; X_{5ac} is 91, 92, 93, 94, 95, 96, 97, 98, 99, or 100; X_{6ac} is 114, 115, or 116; X₁ₐₑ is 22, 23, 24, 25, 26, 27, 28, 29, 30, or 31; X₂ₐₑ is 41, 42, 43, 44, 45, 46, 47, 48, 49, 50, or 51; X₃ₐₑ is 55, 51, 52, 53, 54, 55, 56, 57, 58, 59, 60, 61, 62, 63, 64, 65, 66, 67, 68, 69, 70, 71, 72, 73, or 74; X₄ₐₑ is 77, 78, 79, 80, 81, 82, 83, 84, or 85; X₅ₐₑ is 89, 90, 91, 92, 93, 94, 95, 96, 97, or 98; and X₆ₐₑ is 112, 113, or 114; and wherein the chimeric polypeptide is capable of binding to one or more of Transforming Growth Factor-beta (TGF-β) superfamily members; or one or more of TGF-β receptors.

2. The composition of as set forth in 1, wherein said liver disease is any one of liver diseases, disorders, or damages where modulating TGF-beta activity provides a therapeutic benefit.

3. The composition of as set forth in 1, wherein said liver disease is non-alcoholic fatty liver disease, liver fibrosis, or hepatic inflammation.

4. The composition of as set forth in 1, wherein the sequence of said polypeptide is described by an algorithm 1n2n3n4n5n6n, wherein said 1n, 2n, 3n, 4n, 5n, and 6n represent respectively the first, second, third, fourth, fifth, and sixth domain; and said n is either a or b, and wherein said a represents an amino acid sequence derived from the sequence of SEQ ID NO:2; and said b represents an amino acid sequence derived from any one selected from the group consisting of SEQ ID NO:4, SEQ ID NO:6, SEQ ID NO:8, and SEQ ID NO:10.

5. The composition of as set forth in 4, wherein the sequence of said polypeptide is described by an algorithm 1b2a3b4b5a6a.

6. The composition of as set forth in 1, wherein said first domain comprises amino acid residues 1 to 47 of SEQ ID NO:2; said second domain comprises amino acid residues 28 to 45 of SEQ ID NO:4; said third domain comprises amino acid residues 66 to 85 of SEQ ID NO:2; said fourth domain comprises amino acid residues 86 to 99 of SEQ ID NO:2; said fifth domain comprises amino acid residues 84 to 95 of SEQ ID NO:4; and said sixth domain comprises amino acid residues 96 to 116 of SEQ ID NO:4.

7. The composition of as set forth in 1, wherein said polypeptide comprises the sequence as set forth in SEQ ID NO:12.

8. The composition of as set forth in 1, wherein said polypeptide has at least 95% sequence identity to the sequence as set forth in SEQ ID NO:12.

9. The composition of as set forth in 1, wherein said chimeric polypeptide has at least 97% sequence identity to the amino acid sequence comprising said first domain, said second domain, said third domain, said fourth domain, said fifth domain, and said sixth domain.

10. The composition of as set forth in 1, 2, 3, 4, 5, 6, 7, 8, or 9, wherein the said polypeptide forms a homo-dimer.

11. The composition of as set forth in 1, 2, 3, 4, 5, 6, 7, 8, or 9, wherein the said polypeptide forms a hetero-dimer.

12. A method for treating a liver disease comprising administering a therapeutically effective amount of a composition comprising the chimeric polypeptide of as set forth in 1 to a subject in need thereof.

### Advantageous Effects of Invention

The chimeric polypeptide of an embodiment can refold efficiently and can be produced at a high yield.

The composition of an embodiment can be used to treat any number of liver disease or disorders where modulating of TGF-beta activity provides a therapeutic benefit. For example, the composition of an embodiment can be used in subjects suffering from liver disease, including, but not limited to, non-alcoholic fatty liver disease, liver fibrosis and hepatic inflammation.

### BRIEF DESCRIPTION OF DRAWINGS

FIG. 1 shows the strategies used in an embodiment for generating activin/BMP-6 chimeras, such as AB604, using segments from two different ligands in TGF-beta superfamily.

FIG. 2 shows sequence comparison of TGF-beta superfamily members.

FIG. 3 shows refolding efficiency of BMP-2, 3, 6 and 7.

FIG. 4 shows regions with sequence identity between BMP-6 and Activin which were identified as putative cross-over points.

FIG. 5 shows amino acid sequence of AB604.

FIG. 6 shows aspects of NAFLD and liver fibrosis.

FIG. 7 shows the effects of TGFβ1 on fibronectin and type I collagen gene expression in LX-2 cells.

FIG. 8 shows the effects of TGFβ1 on ALK1 gene expression in LX-2 cells.

FIG. 9 shows the effects of AB604 on suppression of the TGF β1-induced α-SMA gene expression.

FIG. 10 shows the effects of AB604 on suppression of the TGF β1-induced Collagen1 (COL1) and fibronectin gene expression.

FIG. 11 shows inhibition of signaling activity of TGFβ1 by BMP6.

FIG. 12 shows inhibition of signaling activity of TGFβ1 by BMP6 in the presence of 3.0 ng/ml of TGFβ1.

FIG. 13 shows inhibition of signaling activity of TGFβ1 by AB604.

FIG. 14 shows inhibition of signaling activity of TGFβ1 by AB604 in the presence of 3.0 ng/ml of TGFβ1.

FIG. 15 shows comparison of hepcidin expression regulation by BMP6 and AB604.

FIG. 16 shows comparison of dose-dependent profile of hepcidin gene expression by BMP6 and AB604.

FIG. 17 shows comparison of dose-dependent profile of suppression of TGFβ1-mediated fibronectin production by BMP6 and AB604.

FIG. 18 shows the effects of AB604 on decreasing liver size.

FIG. 19 shows the efficacy of AB604 to inhibit liver fibrosis, tested by measuring liver weight after treatment in CCl4-induced liver fibrosis model mice.

FIG. 20 shows the efficacy of AB604 to inhibit liver fibrosis, tested by measuring normalized liver weight after treatment in CCl4-induced liver fibrosis model mice.

### BEST MODE FOR CARRYING OUT THE INVENTION

An embodiment provides a composition for treating a liver disease (including non-alcoholic fatty liver disease, liver fibrosis and hepatic inflammation), wherein the composition comprise a chimeric polypeptide having TGF-beta activity. The chimeric polypeptide, which is sometimes referred to as recombinant polypeptide, is described below.

An embodiment provides a non-naturally occurring chimeric polypeptide having an activity provided by a TGF-beta family member. The chimeric polypeptide of an embodiment comprises two or more domains or fragments from parental TGF-beta proteins operably linked such that the resulting polypeptide is capable of modulating a pathway associated with a TGF-beta family member. In one embodiment, the pathway is a SMAD pathway or a DAXX (Death-associated protein 6) pathway.

An embodiment of the invention provides designer TGF-beta ligands that can be synthesized by selecting and conjoining different domains of TGF-beta superfamily ligands to construct new ligands (i.e. designer ligands). These novel chimeric ligands possess entirely new protein sequence library that differs from naturally existing TGF-beta superfamily ligands. This approach originates primarily from the recognition of the structural commonality among natural TGF-beta superfamily ligands. All ˜40 TGF-beta superfamily ligands share the same overall architecture with generic characteristics for each region of the protein. The framework of TGF-beta ligands can be divided into (generally) six domains that all superfamily members share.

An embodiment of the invention provides a chimeric polypeptide comprising: at least two domains, a first domain of the polypeptide comprising a sequence having at least 80% identity to a first TGF-beta family protein and a second domain comprising a sequence having at least 80% identity to a second TGF-beta family protein, wherein the domains are operably linked and have activity of at least one of the first or second parental TGF-beta family protein. In one embodiment, the chimeric polypeptide comprises 6 domains operably linked N-terminus to C-terminus. In one embodiment, each of the first and second TGF-beta family proteins has structural similarity and each domain corresponds to a structural motif. In one embodiment, the first TGF-beta family protein is BMP-6 and the second TGF-beta family protein is activin. In one embodiment, the polypeptide comprises an N-terminal domain from BMP-6.

In one embodiment, the domains of BMP-6, activin-βA, activin-βB, activin-βC, and activin-βE are as described in Table 1, wherein the chimeric polypeptide has an order of domain 1-domain 2-domain 3-domain 4-domain 5-domain 6, from the N-terminal to the C-terminal orientation.

An embodiment of the invention provides a chimeric polypeptide comprising at least 80%, 81%, 82%, 83%, 84%, 85%, 86%, 86%, 87%, 88%, 89%, 90%, 91%, 92%, 93%, 94%, 95%, 96%, 97%, 98%, 99% or more identity to a sequence as set forth in SEQ ID NO:12 and wherein the polypeptide modulates the SMAD or DAXX pathway.

An embodiment of the invention provides a chimeric TGF-beta family polypeptide comprising a domain of a first TGF-beta family protein operably linked to a domain of a second different TGF-beta family protein to provide a chimeric polypeptide having SMAD or DAXX modulating activity. In one embodiment, the chimeric TGF-beta family polypeptide comprises at least 80%, 81%, 82%, 83%, 84%, 85%, 86%, 86%, 87%, 88%, 89%, 90%, 91%, 92%, 93%, 94%, 95%, 96%, 97%, 98%, 99% or more identity to the sequence as set forth in SEQ ID NO:12 and wherein the polypeptide modulates the SMAD or DAXX pathway.

An embodiment of the invention provides a polynucleotide encoding a polypeptide of an embodiment. In one embodiment, the polynucleotide has at least 80%, 81%, 82%, 83%, 84%, 85%, 86%, 86%, 87%, 88%, 89%, 90%, 91%, 92%, 93%, 94%, 95%, 96%, 97%98%, 99% or more identity to a sequence as set forth in SEQ ID NO:11. A vector comprising such polynucleotide is also provided along with a recombinant cell.

An embodiment of the invention provides novel ligands of the TGF-beta superfamily, wherein each of the ligands is a chimeric protein with at least one of six domains from a foreign or different member of the TGF-beta superfamily.

As used herein and in the appended claims, the singular forms “a,” “and,” and “the” include plural referents unless the context clearly dictates otherwise. Thus, for example, reference to “a domain” includes a plurality of such domains and reference to “the protein” includes reference to one or more proteins, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Although methods and materials similar or equivalent to those described herein can be used in the practice of the disclosed methods and compositions, the exemplary methods and materials are described herein.

Unless defined otherwise, all technical and scientific terms used herein have the same meaning as commonly understood to one of ordinary skill in the art to which this disclosure belongs. Thus, as used throughout the instant application, the following terms shall have the following meanings.

“Amino acid” is a molecule having the structure wherein a central carbon atom (the alpha-carbon atom) is linked to a hydrogen atom, a carboxylic acid group (the carbon atom of which is referred to herein as a “carboxyl carbon atom”), an amino group (the nitrogen atom of which is referred to herein as an “amino nitrogen atom”), and a side chain group, R. When incorporated into a peptide, polypeptide, or protein, an amino acid loses one or more atoms of its amino acid carboxylic groups in the dehydration reaction that links one amino acid to another. As a result, when incorporated into a protein, an amino acid is referred to as an “amino acid residue.”

“Protein” or “polypeptide” refers to any polymer of two or more individual amino acids (whether or not naturally occurring) linked via a peptide bond, and occurs when the carboxyl carbon atom of the carboxylic acid group bonded to the alpha-carbon of one amino acid (or amino acid residue) becomes covalently bound to the amino nitrogen atom of amino group bonded to the -carbon of an adjacent amino acid. The term “protein” is understood to include the terms “polypeptide” and “peptide” (which, at times may be used interchangeably herein) within its meaning. In addition, proteins comprising multiple polypeptide subunits (e.g., DNA polymerase III, RNA polymerase II) or other components (for example, an RNA molecule, as occurs in telomerase) will also be understood to be included within the meaning of “protein” as used herein. Similarly, fragments of proteins and polypeptides are also within the scope of the present invention and may be referred to herein as “proteins.” In one aspect of an embodiment, a polypeptide comprises a chimera of two or more parental domains.

As used herein, TGF-beta superfamily member refers to a TGF-beta superfamily (including bone morphogenic factors) gene or protein of any species, particularly a mammalian species, including but not limited to bovine, ovine, porcine, murine, equine, and human. “TGF-beta superfamily polypeptide” refers to the amino acid sequences of purified TGF-beta superfamily protein obtained from any species, particularly a mammalian species, including bovine, ovine, porcine, murine, equine, and human and from any source, whether natural, synthetic, semi-synthetic, or recombinant.

A particular amino acid sequence of a given protein (i.e., the polypeptide's “primary structure,” when written from the amino-terminus to carboxy-terminus) is determined by the nucleotide sequence of the coding portion of a mRNA, which is in turn specified by genetic information, typically genomic DNA (including organelle DNA, e.g., mitochondrial or chloroplast DNA). Thus, determining the sequence of a gene assists in predicting the primary sequence of a corresponding polypeptide and more particular the role or activity of the polypeptide or proteins encoded by that gene or polynucleotide sequence.

“Fused,” “operably linked,” and “operably associated” are used interchangeably herein to broadly refer to a chemical or physical coupling of two otherwise distinct domains, wherein each domain has independent biological function. As such, an embodiment of the invention provides TGF-beta (e.g., BMP-6 or activin) domains that are fused to one another such that they function as a polypeptide having a TGF-beta family activity or an improvement or change in ligand specificity of a TGF-beta family of polypeptides. In one embodiment, a chimeric polypeptide comprising a plurality of domains from two parental TGF-beta family polypeptides are linked such that they are part of the same coding sequence, each domain encoded by a polynucleotide from a parental TGF-beta family polypeptide, wherein the polynucleotide is in frame such that the polynucleotide when transcribed encodes a single mRNA that when translated comprises a plurality of domains as a single polypeptide. Typically, the coding domains will be linked “in-frame” either directly or separated by a peptide linker and encoded by a single polynucleotide. Various coding sequences for peptide linkers and peptide are known in the art.

“Polynucleotide” or “nucleic acid” refers to a polymeric form of nucleotides. In some instances a polynucleotide comprises a sequence that is not immediately contiguous with either of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an autonomously replicating plasmid or virus; or into the genomic DNA of a prokaryote or eukaryote, or which exists as a separate molecule (e.g., a cDNA) independent of other sequences. The nucleotides of an embodiment can be ribonucleotides, deoxyribonucleotides, or modified forms of either nucleotide. A polynucleotide as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. The term polynucleotide encompasses genomic DNA or RNA (depending upon the organism, i.e., RNA genome of viruses), as well as mRNA encoded by the genomic DNA, and cDNA.

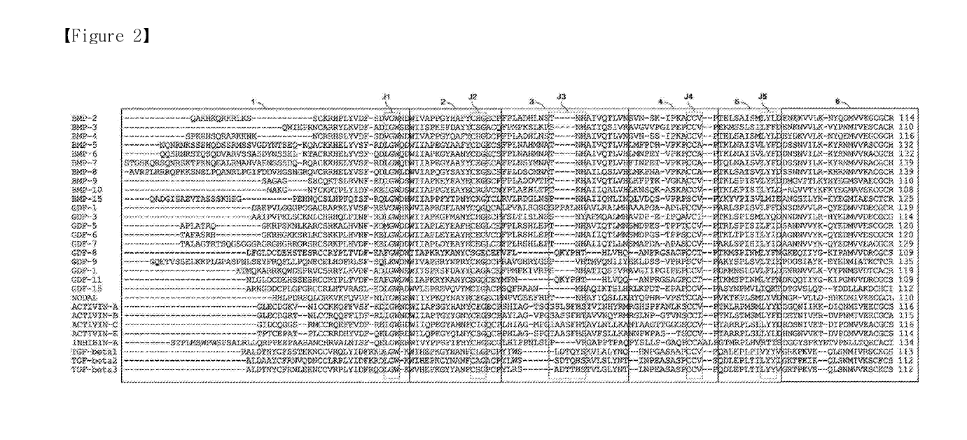

“Chimera” or “chimeric protein” or “chimeric polypeptide” refers to a combination of at least two domains of at least two different parent proteins. For example, a chimeric BMP will have at least two domains from two different parent BMPs; or BMP and other member of the TGF-beta superfamily (for example, activins), or alternatively, an unrelated protein. A chimeric protein may also be an “interspecies,” “intergenic,” etc. fusion of protein structures (the same or different member protein) expressed by different kinds of organisms. In one embodiment, two domains are connected so as to result in a new chimeric protein. In other words, a protein will not be a chimera if it has the identical sequence of either one of the full-length parents. A chimeric protein can comprise more than two domains from two different parent proteins. For example, there may be 2, 3, 4, 5, 6 or 10-20, or more parents from which the domains may be derived in generating a final chimera or library of chimeras. The domain of each parent protein can be very short or very long, the domains can range in length of contiguous amino acids from 1 to about the full length of the protein. In one embodiment, the minimum length is 5 amino acids. Generally, the domain, is one of six domains, alternatively five domains (see FIG. 2).

An embodiment provides a composition for treating a liver disease (including non-alcoholic fatty liver disease, liver fibrosis and hepatic inflammation), wherein the composition comprises a chimeric polypeptide having TGF-beta activity.

In one embodiment, the composition comprises an activin/BMP-6 chimeric polypeptide.

The chimeric polypeptide of an embodiment is described below.

The six domains of a TGF-beta superfamily member are identified based on the structural architecture of the member protein and/or the primary amino acid sequence as aligned against other homologous member proteins. As identified, the member protein is generally divided into 6 distinct domains (although, alternatively, 5 distinct domains) based on domains derived to minimize alterations, or alternatively viewed, maximize alterations, to the aligned native TGF-beta member sequence during chimera engineering. Generally, FIG. 2 shows the relative positions of the distinct domains overlapping the aligned sequences of each of several TGF-beta superfamily members. The vertical line denotes a general position for cross-over between domains in generating the chimera. The amino acids that can overlap the two domains can be defined as being plus or minus about 5 amino acids (or alternatively, 8, 7, 6, 5, 4, 3, 2 or 1 amino acids) in either direction of the vertical line. Also in FIG. 2 is shown a boxed set of amino acids that identify additional junctions that can be used to generate chimera. The J1-J5 junctions are positions general conservation across the TGF-beta family proteins that can be used to generate cross-over points.

Although relatively distinct, the domains may comprise a particular amino acid sequence or an original amino acid sequence that is amenable to substitution(s), insertion(s), additional amino acid(s) at either or both termini of the original sequence, or other modifications. By “amenable”, it is meant that the structural integrity of each domain is maintained as compared to the domain of the original sequence. For example, a domain described herein of a TGF-beta superfamily member may shift by 10, 5, 3, 2, or 1, or preferably no more than 1 amino acid on either or both termini of domain as identified. An embodiment of the invention provides a chimeric protein comprising a fusion of at least one domain from a TGF-beta member with a second domain from a second TGF-beta member, wherein the first domain is foreign to the second TGF-beta member. Utilizing the five-six domains on a single subunit of the TGF-beta superfamily ligand as a scaffold framework, new (designer) sequences can be recombinantly linked by mixing domains from different TGF-beta ligands in the same order as they appear in nature. This assembly produces new sequences that are partly similar to one of several different target sequences, but distinctly different from any naturally occurring sequences.

In one embodiment, a single crossover point is defined for the amino acid sequences of two parents. The crossover location defines where one parent's domain will stop and where the next parent's domain will start. Thus, a simple chimera would only have one crossover location where the domain before that crossover location would belong to one parent and the domain after that crossover location would belong to the second parent. In one embodiment, the chimera has more than one crossover location. For example, 2, 3, 4, 5, 6, 7, 8, 9, 10, 11-30, or more crossover locations. In one embodiment, the parental strands are defined as having 1, 2, 3, 4 or 5 crossover locations. How these crossover locations are named and defined are both discussed below. In an embodiment where there are two crossover locations and two parents, there will be a first contiguous domain from a first parent, followed by a second contiguous domain from a second parent, followed by a third contiguous domain from the first parent. Contiguous is meant to denote that there is nothing of significance interrupting the domains. These contiguous domains are connected to form a contiguous amino acid sequence. For example, an activin/BMP-6 chimera derived from a BMP-6 wild-type parental strand and an activin wild-type parental strand with five crossovers would comprise a first domain from either BMP-6 or activin, a second domain from the opposite parental strand compared to the first domain operably linked to the first domain and comprising a structural motif downstream of the first domain, a third domain from the opposite parental strand compared to the second domain operably linked to the second domain and comprising a structural motif downstream of the second domain, a fourth domain from the opposite parental strand compared to the third domain operably linked to the third domain and comprising a structural motif downstream of the third domain, a fifth domain from the opposite parental strand compared to the fourth domain operably linked to the fourth domain and comprising a structural motif downstream of the fourth domain, and a sixth domain from the opposite parental strand compared to the fifth domain operably linked to the fifth domain and comprising a structural motif downstream of the fifth domain, all connected in one contiguous amino acid chain.

As appreciated by one of skill in the art, variants of chimeras exist as well as the exact sequences. In other words conservative amino acid substitutions may be incorporated into the chimera (e.g., from about 1-10 conservative amino acid substitutions). Thus, not 100% of each domain need be present in the final chimera if it is a variant chimera. The amount that may be altered, either through additional residues or removal or alteration of residues will be defined as the term variant is defined. Of course, as understood by one of skill in the art, the above discussion applies not only to amino acids but also nucleic acids which encode for the amino acids.

“Conservative amino acid substitution” refers to the interchangeability of residues having similar side chains, and thus typically involves substitution of the amino acid in the polypeptide with amino acids within the same or similar defined class of amino acids. By way of example and not limitation, an amino acid with an aliphatic side chain may be substituted with another aliphatic amino acid, e.g., alanine, valine, leucine, isoleucine, and methionine; an amino acid with hydroxyl side chain is substituted with another amino acid with a hydroxyl side chain, e.g., serine and threonine; an amino acids having aromatic side chains is substituted with another amino acid having an aromatic side chain, e.g., phenylalanine, tyrosine, tryptophan, and histidine; an amino acid with a basic side chain is substituted with another amino acid with a basis side chain. e.g., lysine, arginine, and histidine; an amino acid with an acidic side chain is substituted with another amino acid with an acidic side chain. e.g., aspartic acid or glutamic acid; and a hydrophobic or hydrophilic amino acid is replaced with another hydrophobic or hydrophilic amino acid, respectively.

“Non-conservative substitution” refers to substitution of an amino acid in the polypeptide with an amino acid with significantly differing side chain properties. Non-conservative substitutions may use amino acids between, rather than within, the defined groups and affects (a) the structure of the peptide backbone in the area of the substitution (e.g., proline for glycine) (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

“Reference sequence” refers to a defined sequence used as a basis for a sequence comparison. A reference sequence may be a subset of a larger sequence, for example, a domain of a full-length gene or polypeptide sequence. Generally, a reference sequence can be at least 20 nucleotide or amino acid residues in length, at least 25 residues in length, at least 50 residues in length, or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptides are typically performed by comparing sequences of the two polynucleotides or polypeptides over a “comparison window” to identify and compare local regions of sequence similarity.

“Sequence identity” means that two amino acid sequences are substantially identical (i.e., on an amino acid-by-amino acid basis) over a window of comparison. The term “sequence similarity” refers to similar amino acids that share the same biophysical characteristics. The term “percentage of sequence identity” or “percentage of sequence similarity” is calculated by comparing two optimally aligned sequences over the window of comparison, determining the number of positions at which the identical residues (or similar residues) occur in both polypeptide sequences to yield the number of matched positions, dividing the number of matched positions by the total number of positions in the window of comparison (i.e., the window size), and multiplying the result by 100 to yield the percentage of sequence identity (or percentage of sequence similarity). With regard to polynucleotide sequences, the terms sequence identity and sequence similarity have comparable meaning as described for protein sequences, with the term “percentage of sequence identity” indicating that two polynucleotide sequences are identical (on a nucleotide-by-nucleotide basis) over a window of comparison. As such, a percentage of polynucleotide sequence identity (or percentage of polynucleotide sequence similarity. e.g., for silent substitutions or other substitutions, based upon the analysis algorithm) also can be calculated. Maximum correspondence can be determined by using one of the sequence algorithms described herein (or other algorithms available to those of ordinary skill in the art) or by visual inspection.

As applied to polypeptides, the term substantial identity or substantial similarity means that two peptide sequences, when optimally aligned, such as by the programs BLAST, GAP or BESTFIT using default gap weights or by visual inspection, share sequence identity or sequence similarity. Similarly, as applied in the context of two nucleic acids, the term substantial identity or substantial similarity means that the two nucleic acid sequences, when optimally aligned, such as by the programs BLAST, GAP or BESTFIT using default gap weights (described in detail below) or by visual inspection, share sequence identity or sequence similarity.

One example of an algorithm that is suitable for determining percent sequence identity or sequence similarity is the FASTA algorithm, which is described in Pearson, W. R. & Lipman, D. J., (1988) Proc. Natl. Acad. Sci. USA 85:2444. See also, W. R. Pearson, (1996) Methods Enzymology 266:227-258. Preferred parameters used in a FASTA alignment of DNA sequences to calculate percent identity or percent similarity are optimized, BL50 Matrix 15: 5, k-tuple=2; joining penalty=40, optimization=28; gap penalty 12, gap length penalty=2; and width=16.

Another example of a useful algorithm is PILEUP. PILEUP creates a multiple sequence alignment from a group of related sequences using progressive, pairwise alignments to show relationship and percent sequence identity or percent sequence similarity. It also plots a tree or dendogram showing the clustering relationships used to create the alignment. PILEUP uses a simplification of the progressive alignment method of Feng & Doolittle, (1987) J. Mol. Evol. 35:351-360. The method used is similar to the method described by Higgins & Sharp, CABIOS 5:151-153, 1989. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity (or percent sequence similarity) relationship using the following parameters: default gap weight (3.00), default gap length weight (0.10), and weighted end gaps. PILEUP can be obtained from the GCG sequence analysis software package, e.g., version 7.0 (Devereaux et al., (1984) Nuc. Acids Res. 12:387-395).

Another example of an algorithm that is suitable for multiple DNA and amino acid sequence alignments is the CLUSTALW program (Thompson, J. D. et al., (1994) Nuc. Acids Res. 22:4673-4680). CLUSTALW performs multiple pairwise comparisons between groups of sequences and assembles them into a multiple alignment based on sequence identity. Gap open and Gap extension penalties were 10 and 0.05 respectively. For amino acid alignments, the BLOSUM algorithm can be used as a protein weight matrix (Henikoff and Henikoff, (1992) Proc. Natl. Acad. Sci. USA 89:10915-10919).

FIG. 2, for example, shows an alignment of a number of TGF-beta family members. One of skill in the art can readily determine from the alignment those amino acids that are conserved across the family as well as those that are not conserved.

“Functional” refers to a polypeptide which possesses either the native biological activity of the naturally-produced proteins of its type, or any specific desired activity, for example as judged by its ability to bind to ligand or cognate molecules or induce a particular biological function (e.g., stimulate muscle growth, bone growth and the like).

The Transforming Growth Factor-beta (TGF-β) superfamily of proteins is comprised of extracellular cytokines found in the vast majority of human cells. The TGF-β superfamily ligands, of which there are ˜40, can be subdivided into smaller families including TGF-β, Bone Morphogenetic Proteins (BMPs), activin and inhibin. Growth and Differentiation Factors (GDFs), Nodal, Mullerian Inhibiting Substance (MIS), and Glial cell line-Derived Neurotrophic Factors (GDNFs). TGF-β superfamily members are found in a diverse range of cell types and play roles in many fundamental cellular events including dorsal/ventral patterning and left/right axis determination to bone formation and tissue repair. More recently, several TGF-β ligands have been shown to be involved in the maintenance or direct the differentiation of stem cells. Due to their pervasiveness, regulation of TGF-β ligand signaling holds promise for the treatment of a wide range of different diseases from skeletal and muscle abnormalities to numerous neoplastic events. Exemplary sequences are provided herein for various members of this family or proteins, however, one of skill in the art can easily identify homologs and variants using publicly available databases by word search or sequence BLAST searches.

There are generally recognized several subfamilies within the superfamily of TGF-beta (TGF-β1-β5) as well as the differentiation factors (e.g., Vg-1), the hormones activin and inhibin, the Mullerian inhibiting substance (MIS), osteogenic and morphogenic proteins (e.g., OP-1, OP-2, OP-3, other BMPs), the developmentally regulated protein Vgr-1, the growth/differentiation factors (e.g., GDF-1, GDF-3, GDF9 and dorsalin-1), etc. See, e.g., Spam and Roberts (1990) in Peptide Growth Factors and Their Receptors, Spom and Roberts, eds., Springer-Verlag: Berlin pp. 419-472; Weeks and Melton (1987) Cell 51: 861-867; Padgett et al. (1987) Nature 325: 81-84; Mason et al. (1985) Nature 318: 659-663; Mason et al. (1987) Growth Factors 1: 77-88; Cate et al. (1986) Cell 45: 685-698; PCT/US90/05903; PCT/US91/07654; PCT/WO94/10202; U.S. Pat. Nos. 4,877,864; 5,141,905; 5,013,649; 5,116,738; 5,108,922; 5,106,748; and U.S. Pat. No. 5,155,058; Lyons et al. (1989) Proc. Natl. Acad. Sci. USA 86: 4554-58; McPherron et al. (1993) J. Biol. Chem. 268: 3444-3449; Basler et al. (1993) Cell 73: 687-702.

Morphogenic proteins of the TGF-beta superfamily include the mammalian osteogenic protein-1 (OP-1, also known as BMP-7), osteogenic protein-2 (OP-2, also known as BMP-8), osteogenic protein-3 (053), BMP-2 (also known as BMP-2A or CBMP-2A, and the Drosophila homolog DPP), BMP-3, BMP-4 (also known as BMP2B or CBMP-2B), BMP-5, BMP-6 and its murine homolog Vgr-1, BMP-9, BMP-10, BMP-1, BMP-12, GDF3 (also known as Vgr2), GDF-8, GDF-9, GDF-10, GDF-11, GDF-12, BMP-13, BMP-14, BMP-15, GDF-5 (also known as CDMP-1 or MP52), GDF-6 (also known as CDMP-2 or BMP13), GDF-7 (also known as CDMP-3 or BMP-12), the Xenopus homolog Vgl and NODAL, UNIVIN, SCREW, ADMP, NEURAL, etc.

TGF-β ligands are synthesized as inactive precursor molecules composed of an N-terminal pro-domain and a C-terminal mature domain linked by a protease cleavage site. To be become active, the mature domain must be cleaved from the pro-domain, commonly by a convertase, such as furin. Members of the TGF-β superfamily are classified together due to the conserved structural architecture found in their mature domains. In general, each mature ligand monomer contains 7 cysteines, 6 of which form three intra-disulfide bonds arranged in a ‘cystine knot’ motif. Stretching outward from the ‘cystine knot’ are 4 beta strands, creating 2 curved fingers. The last remaining cysteine forms an inter-disulfide bond with a second ligand monomer, generating a covalently linked dimer. The dimer has the overall appearance of a butterfly with the ‘cystine knot’ as the body and the fingers spreading out like wings. The functional subunit for the TGF-β superfamily is the dimer and they been shown to exist both as homo- and heterodimers in vivo. Some family members, such as GDF-9 and BMP-15, lack the cysteine required to form the inter-disulfide bond yet they are still able to form stable dimers.

To initiate the signaling process, TGF-β dimers must recruit two sets of receptors, termed type I and type II. These receptors are serine/threonine kinases possessing an extracellular domain (ECD) ordered into a three-finger toxin fold, a single transmembrane domain, and a large intracellular kinase domain. TGF-β ligands have been shown to display preferences in their affinity for the different receptor types. Activin and Nodal exhibit high affinity for type II receptors, while BMP-2 and GDF-5 possess higher affinity for type I receptors. Following the binding of two high affinity receptors to a TGF-β ligand, two lower affinity receptors are then able to bind and join the complex. Upon binding of all four receptors to the TGF-β ligand, forming a 6-member ternary complex, the downstream signaling cascade is initiated. The constitutively active type II receptors phosphorylate the type I receptors which, in turn, bind and phosphorylate intracellular signaling molecules called Smads. The Smad molecules then are able to translocate to the nucleus and interact directly with transcriptional regulators. Multiple mechanisms are employed to closely regulate TGF-β signaling at different stages of the cascade: Extracellular antagonists, including Noggin, follistatin, and Inhibin; pseudo-receptors lacking the intracellular kinase domain, similar to BAMBI; or through intracellular molecules, such as inhibitory Smads.

TGF-β superfamily shows a high degree of promiscuity by receptors for the ligands. While there are over 40 TGF-β ligands, there are only 12 receptors (7 type I and 5 type II). Therefore, receptors must be able to interact with a multitude of different ligands. For instance, ActRII is known to bind activin and BMP-7 with high affinity, but binds BMP-2 with much lower affinity. In GDF-5, a single amino acid has been found which determines its type I receptor preference, while in BMP-3 a single point mutation was discovered which alters type II receptor affinity as well as imparting function to the ligand. An embodiment provides methods to create modified TGF-β ligands with novel receptor binding properties thereby diversifying TGF-β ligand function as well as a composition having such activity.

An embodiment demonstrates a TGF-beta signaling complex by utilizing novel ligand constructs. Using synthesized chimeric homo- or heterodimeric ligands, an embodiment provides a composition for use in dissecting the signaling of TGF-beta family proteins. Furthermore, utilizing such ligands allows a method for distinguishing contributions of two type I receptor interfaces from each other, and two type II receptor interfaced each other. The methods and compositions of an embodiment demonstrate a correlation between ligand-receptor affinity, signaling activity, and biological activity. The methods and compositions of an embodiment shed light on the mechanism and requirements of the TGF-beta superfamily signaling complex assembly. In addition the chimeric ligands provide novel polypeptides for use in treating diseases and disorders associated with TGF-β family of proteins.

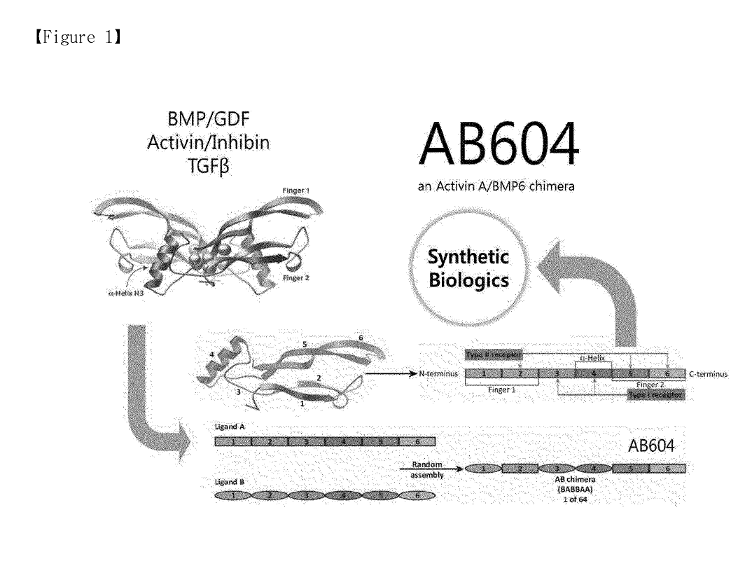

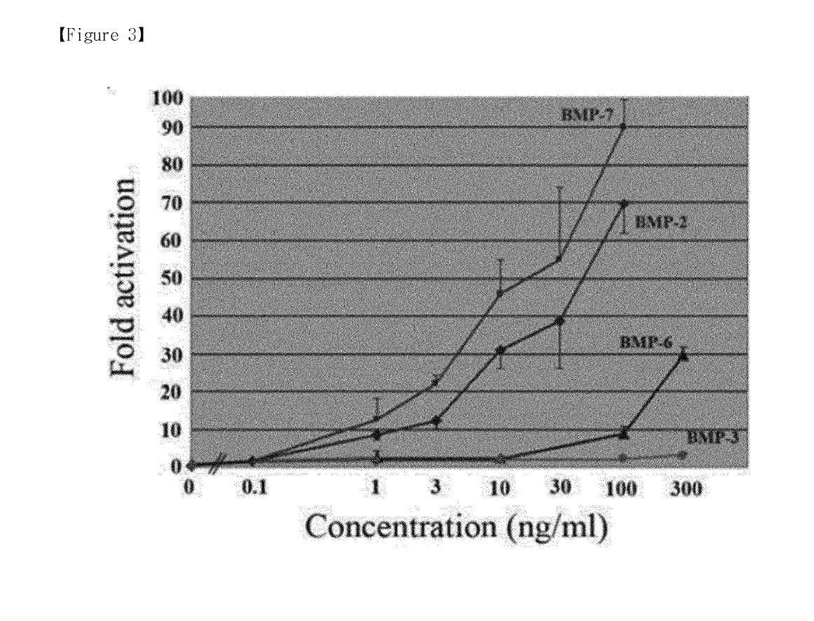

An embodiment provides methods of making and using a novel chimeric TGF-β ligand which possesses the ability to be expressed and refolded using, for example, an E. coli or mammalian expression system. The strategies used in an embodiment for generating an activin/BMP-6 chimera, such as AB604, using domains from two different ligands in TGF-beta superfamily are summarized in FIG. 1. The chimera either mimic a specific TGF-β ligand's signaling characteristics or display unique signaling properties not seen in nature. In one embodiment, activin-βA and BMP-6 are used as templates to generate an activin/BMP-6 chimera. The activin/BMP-6 chimera of an embodiment shows 7-fold increase in Smad-1 signaling activity and 10-fold increase in refolding yield, compared to activin/BMP-2 (AB204). These superior properties of the activin/BMP-6 chimera of an embodiment could not have been anticipated before, considering that BMP-2 was believed to have the highest refolding efficiency among BMP proteins and that BMP-2 was also believed to show higher Smad-1 signaling activity compared to BMP-6, as described in Allendorph et al. Biochemistry, 2007:12238-47 (FIG. 3).

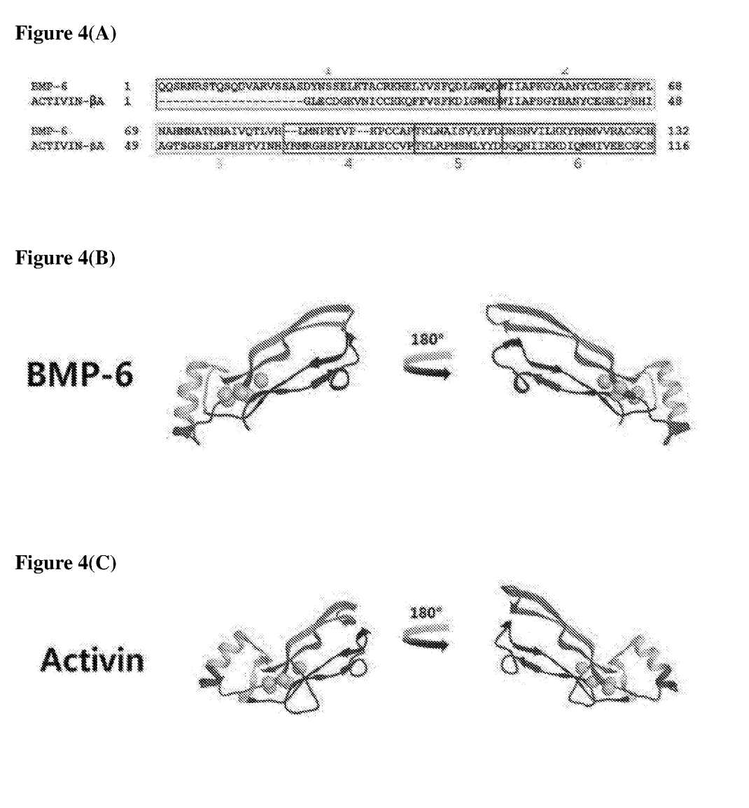

In one embodiment, two factors were considered when looking to design the domains of the chimera. First was a structural consideration. The overall TGF-β monomer fold is divided into 6 domains naturally: Beta strand 1 and 2, the pre-helix loop, alpha helix 1, and beta strand 3 and 4. The identification and characterization of these domains are further described in Example 4. An embodiment utilized chimeric structures to mimic these natural regions in the design. Thus, each domain can be indicated by 1, 2, 3, 4, 5, and 6. The second consideration was to minimize alterations to the aligned native TGF-beta member sequence during chimera engineering. Therefore, those regions with sequence identity between the 2 protein sequences were identified as putative cross-over points. These regions are suitable for the overlaps in DNA sequence for PCR strategy and will minimize any changes to the natural sequences. FIG. 4 illustrates the sequence and structure of these considerations. The regions are boxed and numbered according to their domain and are mapped onto the ligand monomer. Residue numbering in one embodiment is based on BMP-6 (SEQ ID NO:2). Thus, cross-over points in generating a chimeric polypeptide of an embodiment can be identified by identifying similar structural motifs in combination with at least 60%, 70%, 80%, 90%, 95%, 98%, 99% or 100% identity in a domain of the sequence between changes in the structural motif. Cross-overs at these regions (which may be between 3 to 20 amino acids) minimize disruption of the resulting chimeric polypeptide providing a stabilized chimera.

For example, a chimeric polypeptide comprising the algorithm 1b2a3b4b5a6a indicates 6 domains, the letter indicating the parental strand of each domain. Thus, in the example “1b2a3b4b5a6a”, domain 1 is from parental strand “b” for BMP-6, domain 2 is from parental strand “a” for activin, domain 3 is from parental strand “b” for BMP-6, domain 4 is from parental strand “b” for BMP-6, domain 5 is from parental strand “a” for activin, and domain 6 is from parental strand “a” for activin.

In one embodiment, crossover between domains of BMP-6 and a second TGF-beta family protein can occur where structural similarity and sequence similarly overlap. FIG. 4 depicts such an overlap between BMP-6 and activin, wherein crossovers can be generated between about residue D42-P52, G62-S65 T82-H85; K94-T100; and S106-D112 (residue numbering is based on BMP-6 (SEQ ID NO:2)). Sequence alignment of BMP-6 and activin-βA highlight the boundaries of domains 1 through 6. Activin has the extra disulfide bond formed between two Cys.

Other methods for identifying crossover locations may be employed in the generation of the chimeric TGF-beta family polypeptide. For example, SCHEMA is a computational based method for predicting which fragments of homologous proteins can be recombined without affecting the structural integrity of the protein (see, e.g., Meyer et al., (2003) Protein Sci., 12:1686-1693). A chimera with higher stability are identifiable by determining the additive contribution of each domain to the overall stability, either by use of linear regression of sequence-stability data, or by reliance on consensus analysis of the MSAs of folded versus unfolded proteins. SCHEMA recombination ensures that the chimera retain biological function and exhibit high sequence diversity by conserving important functional residues while exchanging tolerant ones.

As presented in an embodiment, it has been found that when these recombined, functional chimeric TGF-beta family polypeptides are generated their ligand specificity can be improved or biological activity can be altered or improved compared to an unrecombined parental polypeptide. Because of differences in activity/ligand profiles, these engineered chimeric TGF-beta family polypeptides provide a unique basis to screen for activities for ligand specific activation and inhibition, provide novel therapeutic polypeptides and research reagents.

For example, in the chimera of an embodiment, domains 1, 2, 3, 4, 5, and 6 can be selected from the following sequences (TABLE 1) wherein the polypeptide comprises a structure (domain 1-domain 2-domain 3-domain 4-domain 5-domain 6):

TABLE 1

| | Parental | |
|---|---|---|
| Amino acids | SEQ ID | |
| (Domain #) | NO: | Variable definition |
| 1-X_{1b} (1) | 2 | X_{1b} is 43, 44, 45, 46, 47, 48, 49, 50, 51, or 52 |
| 1-X₁ₐₐ (1) | 4 | X₁ₐₐ is 22, 23, 24, 25, 26, 27, 28, 29, 30, or 31 |
| 1-X_{1ab} (1) | 6 | X_{1ab} is 22, 23, 24, 25, 26, 27, 28, 29, 30, or 31 |
| 1-X_{1ac} (1) | 8 | X_{1ac} is 22, 23, 24, 25, 26, 27, 28, 29, 30, or 31 |
| 1-X₁ₐₑ (1) | 10 | X₁ₐₑ is 22, 23, 24, 25, 26, 27, 28, 29, 30, or 31 |
| X_{1b}-X_{2b} (2) | 2 | X_{1b} is 43, 44, 45, 46, 47, 48, 49, 50, 51, or 52X_{2b} is 61, 62, 63, |
| | | 64, 65, 66, 67, 68, 69, or 70 |
| X₁ₐₐ-X₂ₐₐ (2) | 4 | X₁ₐₐ is 22, 23, 24, 25, 26, 27, 28, 29, 30, or 31X₂ₐₐ is 41, 42, 43, |
| | | 44, 45, 46, 47, 48, 49, 50, or 51 |
| X_{1ab}-X_{2ab} (2) | 6 | X_{1ab} is 22, 23, 24, 25, 26, 27, 28, 29, 30, or 31X_{2ab} is 41, 42, |
| | | 43, 44, 45, 46, 47, 48, 49, 50, or 51 |
| X_{1ac}-X_{2ac} (2) | 8 | X_{1ac} is 22, 23, 24, 25, 26, 27, 28, 29, 30, or 31X_{2ac} is 41, 42, 43, |
| | | 44, 45, 46, 47, 48, 49, 50, or 51 |
| X₁ₐₑ-X₂ₐₑ (2) | 10 | X₁ₐₑ is 22, 23, 24, 25, 26, 27, 28, 29, 30, or 31X₂ₐₑ is 41, 42, 43, |
| | | 44, 45, 46, 47, 48, 49, 50, or 51 |
| X_{2b}-X_{3b} (3) | 2 | X_{2b} is 61, 62, 63, 64, 65, 66, 67, 68, 69, or 70X_{3b} is 75, 76, 77, |
| | | 78, 79, 80, 81, 82, 83, 84, 85, 86, 87, or 88 |
| X₂ₐₐ-X₃ₐₐ (3) | 4 | X₂ₐₐ is 41, 42, 43, 44, 45, 46, 47, 48, 49, 50, or 51X₃ₐₐ is 55, 51, |
| | | 52, 53, 54, 55, 56, 57, 58, 59, 60, 61, 62, 63, 64, 65, 66, 67, |
| | | 68, 69, 70, 71, 72, 73, or 74 |
| X_{2ab}-X_{3ab} (3) | 6 | X_{2ab} is 41, 42, 43, 44, 45, 46, 47, 48, 49, 50, or 51X_{3ab} is 55, |
| | | 51, 52, 53, 54, 55, 56, 57, 58, 59, 60, 61, 62, 63, 64, 65, 66, |
| | | 67, 68, 69, 70, 71, 72, 73, or 74 |
| X_{2ac}-X_{3ac} (3) | 8 | X_{2ac} is 41, 42, 43, 44, 45, 46, 47, 48, 49, 50, or 51X_{3ac} is 55, 51, |
| | | 52, 53, 54, 55, 56, 57, 58, 59, 60, 61, 62, 63, 64, 65, 66, 67, |
| | | 68, 69, 70, 71, 72, 73, or 74 |
| X₂ₐₑ-X₃ₐₑ (3) | 10 | X₂ₐₑ is 41, 42, 43, 44, 45, 46, 47, 48, 49, 50, or 51X₃ₐₑ is 55, 51, |
| | | 52, 53, 54, 55, 56, 57, 58, 59, 60, 61, 62, 63, 64, 65, 66, 67, |
| | | 68, 69, 70, 71, 72, 73, or 74 |
| X_{3b}-X_{4b} (4) | 2 | X_{3b} is 75, 76, 77, 78, 79, 80, 81, 82, 83, 84, 85, 86, 87, or 88X_{4b} |
| | | is 95, 96, 97, 98, 99, 100, 101, 102, or 103 |
| X₃ₐₐ-X₄ₐₐ (4) | 4 | X₃ₐₐ is 55, 51, 52, 53, 54, 55, 56, 57, 58, 59, 60, 61, 62, 63, 64, |
| | | 65, 66, 67, 68, 69, 70, 71, 72, 73, or 74X₄ₐₐ is 79, 80, 81, 82, |
| | | 83, 84, 85, 86, or 87 |
| X_{3ab}-X_{4ab} (4) | 6 | X_{3ab} is 55, 51, 52, 53, 54, 55, 56, 57, 58, 59, 60, 61, 62, 63, 64, |
| | | 65, 66, 67, 68, 69, 70, 71, 72, 73, or 74X_{4ab} is 78, 79, 80, 81, |
| | | 82, 83, 84, 85, or 86 |
| X_{3ac}-X_{4ac} (4) | 8 | X_{3ac} is 55, 51, 52, 53, 54, 55, 56, 57, 58, 59, 60, 61, 62, 63, 64, |
| | | 65, 66, 67, 68, 69, 70, 71, 72, 73, or 74X_{4ac} is 79, 80, 81, 82, |
| | | 83, 84, 85, 86, or 87 |
| X₃ₐₑ-X₄ₐₑ (4) | 10 | X₃ₐₑ is 55, 51, 52, 53, 54, 55, 56, 57, 58, 59, 60, 61, 62, 63, 64, |
| | | 65, 66, 67, 68, 69, 70, 71, 72, 73, or 74X₄ₐₑ is 77, 78, 79, 80, |
| | | 81, 82, 83, 84, or 85 |
| X_{4b}-X_{5b} (5) | 2 | X_{4b} is 95, 96, 97, 98, 99, 100, 101, 102, or 103X_{5b} is 107, 108, |
| | | 109, 110, 111, 112, 113, 114, or 115 |
| X₄ₐₐ-X₅ₐₐ (5) | 4 | X₄ₐₐ is 79, 80, 81, 82, 83, 84, 85, 86, or 87X₅ₐₐ is 91, 92, 93, 94, |
| | | 95, 96, 97, 98, 99, or 100 |
| X_{4ab}-X_{5ab} (5) | 6 | X_{4ab} is 78, 79, 80, 81, 82, 83, 84, 85, or 86X_{5ab} is 90, 91, 92, |
| | | 93, 94, 95, 96, 97, 98, or 99 |
| X_{4ac}-X_{5ac} (5) | 8 | X_{4ac} is 79, 80, 81, 82, 83, 84, 85, 86, or 87X_{5ac} is 91, 92, 93, 94, |
| | | 95, 96, 97, 98, 99, or 100 |
| X₄ₐₑ-X₅ₐₑ (5) | 10 | X₄ₐₑ is 77, 78, 79, 80, 81, 82, 83, 84, or 85X₅ₐₑ is 89, 90, 91, 92, |
| | | 93, 94, 95, 96, 97, or 98 |
| X_{5b}-X_{6b} (6) | 2 | X_{5b} is 107, 108, 109, 110, 111, 112, 113, 114, or 115X_{6b} is |
| | | 130, 131, or 132 |
| X₅ₐₐ-X₆ₐₐ (6) | 4 | X₅ₐₐ is 91, 92, 93, 94, 95, 96, 97, 98, 99, or 100X₆ₐₐ is 114, 115, |
| | | or 116 |

In some embodiments, domain 3 may be derived from the same parent as either domain 2, domain 4 or both domain 2 and 4.

As summarized in FIG. 2, in some embodiments, J1 (Junction 1) between domain 1 and domain 2 comprises the consensus sequence Z₁Z₂W, wherein Z₁ is selected from the group L, V, F, and M, and Z₂ is G or K, wherein 2 of the 3 amino acids are found at the C-terminus of the first domain or the N-terminus of the second domain. In some embodiment. J2 (Junction 2) between domain 2 and domain 3 comprises the consensus sequence CZ₁G, wherein Z₁ is selected from the group H, S, A, L, I, E, K, Q and D, wherein 2 of the 3 amino acids are found at the C-terminus of the second domain or the N-terminus of the third domain. In some embodiment. J3 (Junction 3) between domain 3 and domain 4 comprises the consensus sequence Z₁Z₂Z₃, wherein Z₁ is selected from the group T, S, P, G and I, Z₂ is selected from the group consisting of N, K, V, M, H and Y, and Z₃ is selected from the group consisting of H, Y, S, T and P, wherein 2 of the 3 amino acids are found at the C-terminus of the third domain or the N-terminus of the fourth domain. In some embodiment, J4 (Junction 4) between domain 4 and domain 5 comprises the consensus sequence Z₁CZ₂, wherein Z₁ is selected from the group C, S and V, and Z₂ is selected from the group consisting of V, A, I and T, wherein 2 of the 3 amino acids are found at the C-terminus of the fourth domain or the N-terminus of the Fifth domain. In some embodiment, J5 (Junction 1) between the domain 5 and domain 6 comprises the consensus sequence Z₁Z₂Z₃, wherein Z₁ is selected from the group L, R and V, Z₂ is selected from the group consisting of T, Q, Y, F and M, and Z₃ is selected from the group consisting of L, F, Y, K, I, Q, V and T, wherein 2 of the 3 amino acids are found at the C-terminus of the fifth domain or the N-terminus of the sixth domain.

An embodiment of the invention provides the following domains (Table 2) for each of the TGF-beta family members that may be recombined to form a chimera of an embodiment having increased or improved biological activity (e.g., resistance to inactivation and the like).

TABLE 2

| | Domain 1 | Domain 2 | Domain 3 | Domain 4 | Domain 5 | Domain 6 |
|---|---|---|---|---|---|---|
| BMP-6(SEQ | 1-47 | 48-65 | 66-85 | 86-99 | 100-111 | 112-132 |
| ID NO: 2) | | | | | | |
| activin-βA(SEQ | 1-27 | 28-45 | 46-68 | 69-83 | 84-95 | 96-116 |
| ID NO: 4) | | | | | | |
| activin-βB(SEQ | 1-27 | 28-45 | 46-68 | 69-82 | 83-94 | 95-115 |
| ID NO: 6) | | | | | | |
| activin-βC(SEQ | 1-27 | 28-45 | 46-68 | 69-83 | 84-95 | 96-116 |
| ID NO: 8) | | | | | | |
| activin-βE(SEQ | 1-27 | 28-45 | 46-68 | 69-81 | 82-93 | 94-114 |
| ID NO: 10) | | | | | | |

Thus, as illustrated by various embodiments herein, an embodiment provides a chimeric TGF-beta family polypeptide, wherein a first TGF-beta family protein (i.e., a first parental protein), which is BMP-6 (SEQ ID NO:2), is recombined with a second different TGF-beta family protein (i.e., a second parental protein), which is activin, to provide a chimeric polypeptide. (SEQ ID NO:1 shows DNA sequence of BMP-6 DNA.)

In some embodiments, the composition comprises a chimeric polypeptide comprising one or more domains of a BMP-6 protein, wherein the domains of BMP-6 are as described in Table 1, such that a contiguous polypeptide comprising domains 1b2b3b4b5b6b comprises a wild-type BMP-6 following a Methionine residue resulting from the translation initiation codon (ATG). Homologs and proteins having at least about 80%, 81%, 82%, 83%, 84%, 85%, 86%, 87%, 88%, 89%, 90%, 91%, 92%, 93%, 94%, 95%, 96%, 97%, 98%, and 99% identity to the foregoing sequences are also included by the present invention.

In some embodiments, the composition comprises a chimeric polypeptide comprising one or more domains of activin proteins, wherein the domains of activin-βA (SEQ ID NO:4), activin-βB (SEQ ID NO:6), activin-βC (SEQ ID NO:8), and activin-βE (SEQ ID NO:10) are as described in Table 1, such that a contiguous polypeptide comprising domains 1a2a3a4a5a6a comprises a wild-type mature activin-βA, activin-βB, activin-βC, or activin-βE protein or a chimera of activin-βA, activin-βB, activin-βC. and activin-βE. Homologs and proteins having at least about 80%, 81%, 82%, 83%, 84%, 85%, 86%, 87%, 88%, 89%, 90%, 91%, 92%, 93%, 94%, 95%, 96%, 97%, 98%, and 99% identity to the foregoing sequences are also included by the present invention. (SEQ ID NO:3, SEQ ID NO:5, SEQ ID NO:7, and SEQ ID NO:9 show DNA sequences of activin-βA, activin-βB, activin-βC, and activin-βE, respectively.)

In other embodiment, the chimeric polypeptide may be fused to an additional heterologous polypeptide to generate a chimeric fusion polypeptide. The heterologous polypeptide may be, for example, a peptide useful for purification or that permits oligomerization of multiple chimeric polypeptides of an embodiment of the present invention. The heterologous may be chemically conjugated to the chimeric polypeptide or may be operably linked in-frame with a coding sequence for the chimeric polypeptide.

In one embodiment, the amino acid sequence of the chimeric polypeptide is described by an algorithm 1n2n3n4n5n6n, wherein said 1n, 2n, 3n, 4n, 5n, and 6n represent respectively the first, second, third, fourth, fifth, and sixth domain; and said n is either a or b, and wherein said a represents an amino acid sequence derived from the sequence of SEQ ID NO:2; and said b represents an amino acid sequence derived from any one selected from the group consisting of SEQ ID NO:4, SEQ ID NO:6, SEQ ID NO:8, and SEQ ID NO:10. For example, the chimeric polypeptide may comprise a sequence described by an algorithm selected from the group consisting of 1a2b3b4b5b6b; 1a2b3b4b5b6a; 1a2b3b4b5a6a; 1a2b3b4b5a6b; 1a2b3b4a5a6a; 1a2b3b4a5b6b; 1a2b3b4a5a6b; 1a2b3b4a5b6a; 1a2b3a4a5a6a; 1a2b3a4a5a6b; 1a2b3a4a5b6b; 1a2b3a4a5b6a; 1a2b3a4b5b6b; 1a2b3a4b5b6a; 1a2b3a4b5a6a; 1a2b3a4b5a6b; 1a2a3a4a5a6a; 1a2a3a4a5a6b; 1a2a3a4a5b6b; 1a2a3a4a5b6a; 1a2a3a4b5b6b; 1a2a3a4b5b6a; 1a2a3a4b5a6b; 1a2a3a4b5a6a; 1a2a3b4b5b6b; 1a2a3b4b5b6a; 1a2a3b4b5a6a; 1a2a3b4b5a6b; 1a2a3b4a5a6a; 1a2a3b4a5b6a; 1a2a3b4a5b6b; 1a2a3b4a5a6b; 1b2b3b4b5b6b; 1b2b3b4b5b6a; 1b2b3b4b5a6a; 1b2b3b4b5a6b; 1b2b3b4a5a6a; 1b2b3b4a5b6b; 1b2b3b4a5a6b; 1b2b3b4a5b6a; 1b2b3a4a5a6a; 1b2b3a4a5a6b; 1b2b3a4a5b6b; 1b2b3a4a5b6a; 1b2b3a4b5b6b; 1b2b3a4b5b6a; 1b2b3a4b5a6a; 1b2b3a4b5a6b; 1b2a3a4a5a6a; 1b2a3a4a5a6b; 1b2a3a4a5b6b; 1b2a3a4a5b6a; 1b2a3a4b5b6b; 1b2a3a4b5b6a; 1b2a3a4b5a6b; 1b2a3a4b5a6a; 1b2a3b4b5b6b; 1b2a3b4b5b6a; 1b2a3b4b5a6a; 1b2a3b4b5a6b; 1b2a3b4a5a6a; 1b2a3b4a5b6a; 1b2a3b4a5b6b; 1b2a3b4a5a6b, wherein said 1b, 2b, 3b, 4b, 5b, and 6b are respectively said first, second, third, fourth, fifth, and sixth segment of SEQ ID NO:2 and said 1a, 2a, 3a, 4a, 5a, and 6a are respectively said first, second, third, fourth, fifth, and sixth segment of any one selected from the group consisting of SEQ ID NO:4, SEQ ID NO:6, SEQ ID NO:8, and SEQ ID NO:10.

In one embodiment, the amino acid sequence of the chimeric polypeptide is described by an algorithm 1b2a3b4b5a6a.

In one embodiment, said first domain comprises amino acid residues 1 to 47 of SEQ ID NO:2; said second domain comprises amino acid residues 28 to 45 of SEQ ID NO:4; said third domain comprises amino acid residues 66 to 85 of SEQ ID NO:2; said fourth domain comprises amino acid residues 86 to 99 of SEQ ID NO:2; said fifth domain comprises amino acid residues 84 to 95 of SEQ ID NO:4; and said sixth domain comprises amino acid residues 96 to 116 of SEQ ID NO:4.

In one embodiment, the amino acid sequence of the chimeric polypeptide comprises the sequence as set forth in SEQ ID NO:12.

In one embodiment, the chimeric polypeptide has at least 95% sequence identity to the sequence as set forth in SEQ ID NO:12.

In one embodiment, the chimeric polypeptide has at least 97% sequence identity to the amino acid sequence comprising said first domain, said second domain, said third domain, said fourth domain, said fifth domain, and said sixth domain.

In one embodiment, the chimeric polypeptide in the composition can form a homo-dimer.

In one embodiment, the chimeric polypeptide in the composition can form a hetero-dimer.

In some embodiments, one or more of the domains of a chimeric polypeptide is 100% identical to the parental strand from which each domain was derived. In other embodiments one or more of the domains can comprise an amino acid sequence that has at least 60%, 70%, 80%, 90%, 91%, 92%, 93%, 94%, 95%, 96%, 97%, 98%, or 99% or more identity to a corresponding domain in a parental strand. For example, one of more of the domains may have one or more conservative amino acid substitutions (e.g., 1-5 conservative amino acid substitutions).

In some embodiments, the chimeric TGF-beta family polypeptide may have improved activity compared to one or more of the parental strands from which the chimeric polypeptide is generated. Biological activity of a chimeric polypeptide of an embodiment can be measured using any number of recognized assays in the art for TGF-beta activity. Such assays include, but are not limited to, BIAcore (Surface Plasmon Resonance); C₂Cl₂ luciferase assay: Smad 1/5 reporter system; HEK293 luciferase assay: Smad 2/3 reporter system; FSH (Follicle Stimulating Hormone) release assay: in rat pituitary cells; BRE (BMP Response Element) luciferase assay: Smad 1/5 reporter HEK 293 cells; Cripto binding assay: Luciferase response measured in presence/absence of Crptio; Human Stem Cell assay: Maintenance or Differentiation of H9 cells; NMR binding Studies; Micro mass culture: Bone formation measured in Chick embryos; X-ray Crystallography: Determine Structure of ligand:receptor complexes; Native Gel: Visualization of ligand:receptor complexes; Size Exclusion Chromatography (SEC): Visualization of ligand:receptor complexes; Velocity Scan Ultracentrifugation: Visualize ligand:receptor complex formation; and Seldi mass Spectrometry: Accurately determine size of ligands.

The chimeric TGF-beta family polypeptide described herein may be prepared in various forms, such as lysates, crude extracts, or isolated preparations.

In some embodiments, the isolated chimeric polypeptide is a substantially pure polypeptide composition. A “substantially pure polypeptide” refers to a composition in which the polypeptide species is the predominant species present (i.e., on a molar or weight basis it is more, abundant than any other individual macromolecular species in the composition), and is generally a substantially purified composition when the object species comprises at least about 50 percent of the macromolecular species present by mole or % weight. Generally, a substantially pure polypeptide composition will comprise about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species.

An embodiment contemplates making functional variants by modifying the structure of the chimera. Such modifications may be made, for example, for such purposes as enhancing therapeutic efficacy, or stability (e.g., ex vivo shelf life and resistance to proteolytic degradation in vivo, improve stability, solubility, bioavailability, or biodistribution of the chimeric protein, etc.). For example, but not by way of limitation, the derivatives include a chimera that has been modified, e.g., by acetylation, carboxylation, acylation glycosylation, pegylation, phosphorylation, farnesylation, biotinylation, lipidation, amidation, derivatization by known protecting/blocking groups, proteolytic cleavage, linkage to a cellular ligand or other protein such as an organic deriatizing agent, etc. Any of numerous chemical modifications may be carried out by known techniques, including, but not limited to specific chemical cleavage, acetylation, formylation, metabolic synthesis, etc. Additionally, the derivative may contain one or more non-natural amino acids, such as those with ketone-containing side chain, polyethylene glycols, lipids, poly- or mono-saccharide, and phosphates. Effects of such non-natural amino acid elements on the functionality of a chimeric TGF-beta superfamily protein may be tested as described herein for other TGF-beta superfamily protein variants. When a chimeric TGF-beta superfamily protein is produced in cells by cleaving a nascent form of the precursor protein, post-translational processing may also be important for correct folding and/or function of the protein. Different cells (such as CHO, HeLa, MDCK, 293, W138, NIH-3T3 or HEK293) have specific cellular machinery and characteristic mechanisms for such post-translational activities and may be chosen to ensure the correct post-translational modification and processing of the precursor protein into a chimeric TGF-beta superfamily protein. In vitro cell-free expression system in combination with its associated engineered tRNA synthase and tRNA can be utilized to ensure the correct modification in a specific amino acid position genetically tagged to introduce non-natural amino acids.

A modified chimera can also be produced, for instance, by amino acid substitution, deletion, or addition. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains.

An embodiment contemplates making mutations in a proteolytic cleavage site of the chimera sequence to make the site less susceptible to proteolytic cleavage. Computer analysis (using a commercially available software, e.g., MacVector, Omega, PCGene, Molecular Simulation, Inc.) can be used to identify proteolytic cleavage sites. As will be recognized by one of skill in the art, most of the described mutations, variants or modifications may be made at the nucleic acid level or, in some cases, by post translational modification or chemical synthesis. Such techniques are well known in the art.

An embodiment contemplates specific mutations of a chimera sequences so as to alter the glycosylation of the chimera. Such mutations may be selected so as to introduce or eliminate one or more glycosylation sites, such as O-linked or N-linked glycosylation sites. Asparagine-linked glycosylation recognition sites generally comprise a tripeptide sequence, asparagine-X-threonine (where “X” is any amino acid) which are specifically recognized by appropriate cellular glycosylation enzymes. The alteration may also be made by the addition of, or substitution by, one or more serine or threonine residues to the sequence of the wild-type polypeptide (for O-linked glycosylation sites). A variety of amino acid substitutions or deletions at one or both of the first or third amino acid positions of a glycosylation recognition site (and/or amino acid deletion at the second position) results in non-glycosylation at the modified tripeptide sequence. Another means of increasing the number of carbohydrate moieties is by chemical or enzymatic coupling of glycosides to the polypeptide. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. These methods are described in WO 87/05330 published Sep. 11, 1987, and in Aplin and Wriston (1981) CRC Crit. Rcv. Biochem., pp. 259-306, incorporated by reference herein. Removal of one or more carbohydrate moieties present on a chimera may be accomplished chemically and/or enzymatically. Chemical deglycosylation may involve, for example, exposure to the compound trifluoromethanesulfonic acid, or an equivalent compound. This treatment results in the cleavage of most or all sugars except the linking sugar (N-acetylglucosamine or N-acetylgalactosamine), while leaving the amino acid sequence intact. Chemical deglycosylation is further described by Hakimuddin et al. (1987) Arch. Biochem. Biophys. 259:52 and by Edge et al. (1981) Anal. Biochem. 118:131. Enzymatic cleavage of carbohydrate moieties on polypeptides can be achieved by the use of a variety of endo- and exo-glycosidases as described by Thotakura et al. (1987) Meth. Enzymol. 138:350. The nucleic acid and/or amino acid sequence of a propeptide may be adjusted, as appropriate, depending on the type of expression system used, as mammalian, yeast, insect and plant cells may all introduce differing glycosylation patterns that can be affected by the amino acid sequence of the peptide.

In some embodiments, the chimeric polypeptide can be in the form of arrays. The polypeptide may be in a soluble form, for example as solutions in the wells of mircotitre plates, or immobilized onto a substrate. The substrate can be a solid substrate or a porous substrate (e.g. membrane), which can be composed of organic polymers such as polystyrene, polyethylene, polypropylene, polyfluoroethylene, polyethyleneoxy, and polyacrylamide, as well as co-polymers and grafts thereof. A solid support can also be inorganic, such as glass, silica, controlled pore glass (CPG), reverse phase silica or metal, such as gold or platinum. The configuration of a substrate can be in the form of heads, spheres, particles, granules, a gel, a membrane or a surface. Surfaces can be planar, substantially planar, or non-planar. Solid supports can be porous or non-porous, and can have swelling or non-swelling characteristics. A solid support can be configured in the form of a well, depression, or other container, vessel, feature, or location. A plurality of supports can be configured on an array at various locations, addressable for robotic delivery of reagents, or by detection methods and/or instruments.

An embodiment also provides a polynucleotide encoding the chimeric TGF-beta family polypeptide disclosed herein. The polynucleotide may be operably linked to one or more heterologous regulatory or control sequences that control gene expression to create a recombinant polynucleotide capable of expressing the polypeptide. Expression constructs containing a polynucleotide encoding the chimeric polypeptide can be introduced into appropriate host cells to express the polypeptide. Polynucleotide sequences encoding various domains or full chimera of an embodiment can be determined without undue efforts based upon the various codons that are associated with an amino acid of in a polypeptide. Furthermore, an embodiment provides exemplary sequences of the TGF-β family member. Deriving the sequences of a domain or chimera from the sequences provided herein is readily performed by one of skill in the art. Given the knowledge of specific sequences of the TGF-beta family of proteins, and the specific descriptions of the chimeric polypeptide herein (e.g., the domain structure of the chimeric domains), the nucleic acid sequence of the engineered chimera will be apparent to the skilled artisan. The knowledge of the codons corresponding to various amino acids coupled with the knowledge of the amino acid sequence of the polypeptide allows those skilled in the art to make different polynucleotides encoding the polypeptide of an embodiment. Thus, an embodiment contemplates each and every possible variation of the polynucleotide that could be made by selecting combinations based on possible codon choices, and all such variations are to be considered specifically disclosed for any of the polypeptides described herein.

In some embodiments, the polynucleotide comprises a polynucleotide that encodes the polypeptide described herein but have about 80% or more sequence identity, about 85% or more sequence identity, about 90% or more sequence identity, about 91% or more sequence identity, about 92% or more sequence identity, about 93% or more sequence identity, about 94% or more sequence identity, about 95% or more sequence identity, about 96% or more sequence identity, about 97% or more sequence identity, about 98% or more sequence identity, or about 99% or more sequence identity at the nucleotide level to a reference polynucleotide encoding a chimera or parental TGF-beta family polypeptide.

In some embodiments, the isolated polynucleotide encoding the polypeptide may be manipulated in a variety of ways to provide for expression of the polypeptide. Manipulation of the isolated polynucleotide prior to its insertion into a vector may be desirable or necessary depending on the expression vector. The techniques for modifying polynucleotides and nucleic acid sequences utilizing recombinant DNA methods are well known in the art. Guidance is provided in Sambrook et al., 2001, Molecular Cloning: A Laboratory Manual, 3rd Ed., Cold Spring Harbor Laboratory Press; and Current Protocols in Molecular Biology, Ausubel. F. ed., Greene Pub. Associates, 1998, updates to 2007.

In some embodiments, the polynucleotide is operatively linked to control sequences for the expression of the polynucleotide and/or polypeptide. In some embodiments, the control sequence may be an appropriate promoter sequence, which can be obtained from genes encoding extracellular or intracellular polypeptides, either homologous or heterologous to the host cell.

In some embodiments, the control sequence may also be a suitable transcription terminator sequence, a sequence recognized by a host cell to terminate transcription. The terminator sequence is operably linked to the 3′ terminus of the nucleic acid sequence encoding the polypeptide. Any terminator which is functional in the host cell of choice may be used.

In some embodiments, the control sequence may also be a suitable leader sequence, a nontranslated region of an mRNA that is important for translation by the host cell. The leader sequence is operably linked to the 5′ terminus of the nucleic acid sequence encoding the polypeptide. Any leader sequence that is functional in the host cell of choice may be used.

In some embodiments, the control sequence may also be a signal peptide coding region that codes for an amino acid sequence linked to the amino terminus of a polypeptide and directs the encoded polypeptide into the cell's secretory pathway. The 5′ end of the coding sequence of the nucleic acid sequence may inherently contain a signal peptide coding region naturally linked in translation reading frame with the domain of the coding region that encodes the secreted polypeptide. Alternatively, the 5′ end of the coding sequence may contain a signal peptide coding region that is foreign to the coding sequence. The foreign signal peptide-coding region may be required where the coding sequence does not naturally contain a signal peptide coding region.

An embodiment is further directed to a recombinant expression vector comprising a polynucleotide encoding the chimeric TGF-beta polypeptide described herein, and one or more expression regulating regions such as a promoter and a terminator, a replication origin, etc., depending on the type of hosts into which they are to be introduced. In creating the expression vector, the coding sequence is located in the vector so that the coding sequence is operably linked with the appropriate control sequences for expression.

The recombinant expression vector may be any vector (e.g., a plasmid or virus), which can be conveniently subjected to recombinant DNA procedures and can bring about the expression of the polynucleotide sequence. The choice of the vector will typically depend on the compatibility of the vector with the host cell or in vitro cell-free reaction mixture into which the vector is to be introduced. The vector may be linear or closed circular plasmids.

The expression vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon, may be used.

In some embodiments, the expression vector contains one or more selectable markers, which permit easy selection of transformed cells. A selectable marker is a gene the product of which provides for biocide or viral resistance, resistance to heavy metals, prototrophy to auxotrophs, and the like.

An embodiment provides a host cell comprising a polynucleotide encoding the chimeric TGF-beta polypeptide, the polynucleotide being operatively linked to one or more control sequences for expression of the fusion polypeptide in the host cell. Host cells for use in expressing the fusion polypeptide encoded by the expression vector of an embodiment are well known in the art. Appropriate culture mediums and growth conditions for the above-described host cells are well known in the art.

An expression vector can be designed for expression of a chimera in prokaryotic or eukaryotic cells. For example, a chimera of an embodiment can be expressed in bacterial or prokaryote cells such as E. Coli, insect cells (e.g., the baculovirus expression system), yeast cells, microalgae, plant cells or mammalian cells as well as in vitro cell-free expression system. Some suitable host cells are discussed further in Goeddel, Gene Expression Technology: Methods in Enzymology 185, Academic Press. San Diego, Calif. (1990).

While one example of an expression system discussed is an E. coli expression system, to those skilled in the art, these proteins can be easily be cloned into and expressed from a large number of other expression systems. The advantages include, but are not limited to, achieving post-translational modifications as would be seen in the organism the protein was derived from (in this case H. sapiens), expression of the ligands without the start methionine required for bacterial expression, and easy incorporation of non-natural amino acids or additional chemical modifications. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635). In addition to prokaryotes, eukaryotic microbes such as filamentous fungi or yeast are suitable cloning or expression hosts for VEGF-E-encoding vectors. Saccharomyces cerevisiae is a commonly used lower eukaryotic host microorganism.

Suitable host cells for the expression of a chimera are derived from unicellular and multicellular organisms. Examples of invertebrate cells include insect cells such as Drosophila S2 and Spodoptera Sf9, as well as plant cells. Plant expression systems have also been used successfully to express modified proteins. Examples of useful mammalian host cell lines include Chinese hamster ovary (CHO) and COS cells. More specific examples include monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); Chinese hamster ovary cells/DHFR(CHO, Urlaub and Chasin, Proc. Natl. Acad. Sci. USA, 77:4216 (1980)); mouse sertoli cells (TM4, Mather, Biol. Reprod., 23:243-251 (1980)); human lung cells (W138, ATCC CCL 75); human liver cells (Hep G2, HB 8065); and mouse mammary tumor (MMT 060562, ATCC CCL51). The selection of the appropriate host cell is deemed to be within the skill in the art.

Alternate protein expression systems include human embryonic kidney (HEK) 293 cells, insect cell line (S. frugiperda) utilizing the baculovirus expression system, yeast expression systems not limited to P. pastoris and S. cerevisiae, and numerous Microalgae strains. Transgenic animals can be used to express correctly modified protein. In essence, the animals become living ‘bioreactors’ capable of expressing large amounts of the desired protein in an easily harvested fluid or tissue, such as the milk from a cow. Cell-free in vitro expression systems using either the bacterial or wheat germ cell lysate can be employed. Cell-free expression system will permit inserting a wide range of non-natural amino acids or epitope tags with higher efficiency and greater specificity.

Examples of bacterial vectors include pQE70, pQE60, pQE-9 (Qiagen), pBS, pD10, phagescript, psiX174, pbluescript SK, pbsks, pNH8A, pNH16a, pNH18A, pNH46A (Stratagene); ptrc99a, pKK223-3, pKK233-3, pDR540, and pRIT5 (Pharmacia). Examples of vectors for expression in the yeast S. cerevisiae include pYepSec1 (Baldari et al., EMBO J. 6:229 (1987)), pMFa (Kurjan and Herskowitz, Cell 30:933 (1982)), pJRY88 (Schultz et al., Gene 54:113 (1987)), and pYES2 (Invitrogen Corporation, San Diego, Calif.). Baculovirus vectors available for expression of nucleic acids to produce proteins in cultured insect cells (e.g., Sf9 cells) include the pAc series (Smith et al., Mol. Cell. Biol. 3:2156 (1983)) and the pVL series (Lucklow and Summers Virology 170:31 (1989)).

Examples of mammalian expression vectors include pWLNEO, pSV2CAT, pOG44, pXT1, pSG (Stratagene) pSVK3, PBPV, pMSG, PSVL (Pharmacia), pCDM8 (Seed, Nature 329:840 (1987)) and pMT2PC (Kaufman et al., EMBO J. 6:187 (1987)). When used in mammalian cells, the expression vector's control functions are often provided by viral regulatory elements. For example, commonly used promoters are derived from polyoma, adenovirus 2, cytomegalovirus and Simian Virus 40.

Viral vectors have been used in a wide variety of gene delivery applications in cells, as well as living animal subjects. Viral vectors that can be used include, but are not limited to, retrovirus, lentivirus, adeno-associated virus, poxvirus, alphavirus, baculovirus, vaccinia virus, herpes virus, Epstein-Barr virus, adenovirus, geminivirus, and caulimovirus vectors. Non-viral vectors include plasmids, liposomes, electrically charged lipids (cytofectins), nucleic acid-protein complexes, and biopolymers. In addition to a nucleic acid of interest, a vector may also comprise one or more regulatory regions, and/or selectable markers useful in selecting, measuring, and monitoring nucleic acid transfer results (delivery to specific tissues, duration of expression, etc.).

The chimera of an embodiment can be made by using methods well known in the art. A polynucleotide can be synthesized by recombinant techniques, such as that provided in Sambrook et al., 2001, Molecular Cloning: A Laboratory Manual, 3rd Ed., Cold Spring Harbor Laboratory Press; and Current Protocols in Molecular Biology, Ausubel. F. ed., Greene Pub. Associates, 1998, updates to 2007. Polynucleotides encoding the enzymes, or the primers for amplification can also be prepared by standard solid-phase methods, according to known synthetic methods, for example using phosphoramidite method described by Beaucage et al., (1981) Tet Lett 22:1859-69, or the method described by Matthes et al., (1984) EMBO J. 3:801-05, e.g., as it is typically practiced in automated synthetic methods. In addition, automated peptide synthesizers are commercially available (e.g., Advanced ChemTech Model 396; Milligen/Biosearch 9600).

An embodiment is directed to a method to accelerate construction of large chimera libraries. Accordingly, an embodiment provides a recombinant strategy termed RASCH (RAndom Segmental CHimera). It uses a template sequence (first strand from one TGF-beta superfamily member) and a few target sequences (second (third, fourth, fifth, sixth) strand from one or more alternate TGF-beta superfamily members), whose domains are to be linked. The template DNA sequence is used to promote efficient coupling of the target sequences and is degraded once domains are linked. Following the gene construction to create the chimeric sequences, the new ligands are chemically refolded into functional dimer. This dimerization process permits additional diversification of the final sequence by mixing and dimerizing two different sequences of both natural and designer origins. Therefore, the RASCH method can be used to diversify the approximate 40 natural protein sequences of TGF-beta superfamily ligands into ten of thousands or more variant sequences, each distinct from any naturally-occurring TGF-beta superfamily ligand sequences.

Engineered polypeptide expressed in a host cell can be recovered from the cells and or the culture medium using any one or more of the well-known techniques for protein purification, including, among others, lysozyme treatment, sonication, filtration, salting-out, ultra-centrifugation, chromatography, and affinity separation (e.g., substrate bound antibodies).

Chromatographic techniques for isolation of the polypeptide include, among others, reversed phase chromatography high performance liquid chromatography, ion exchange chromatography, gel electrophoresis, and affinity chromatography. Conditions for purifying a particular enzyme will depend, in part, on factors such as net charge, hydrophobicity, hydrophilicity, molecular weight, molecular shape, etc., and will be apparent to those having skill in the art.

Assays to determine activity are well known in the art. An embodiment relates to assays to test for biological activity of chimeric proteins, more preferably, to assays to test for clinical activity. Such activity can include enhanced agonistic or antagonistic TGF-beta activity, combined or novel biological activity, and the like.

In certain embodiments, a chimeric protein of an embodiment comprising an agonist of a TGF-beta superfamily protein comprises an antagonist of a different TGF-beta superfamily protein.

Irrespective of which protein expression, harvesting, and, folding methodologies are used, certain of the subject chimeric proteins can bind, preferentially to a pre-selected receptor and can now be identified using standard methodologies, e.g., ligand/receptor binding assays, well known, and thoroughly documented in the art. See, e.g., Legerski gl al. (1992) Bio h_Biophys. Res. Comm. 183: 672679; Frakar et al. (1978) Biochem. Bio12-hys. Res. Comm 80:849-857; Chio et el. (1990) Nature 343: 266-269; Dahlman et al. (1988) Biochem 27: 1813-1817; Strader et el. (1989) J. Biol. Chem. 264: 13572-13578; and DDowd et al. (1988) J. Biol. Chem. 263: 15985-15992.

Typically, in a ligand/receptor binding assay, the native or parent TGF-beta superfamily member of interest having a known, quantifiable affinity for a pre-selected receptor is labeled with a detectable moiety, for example, a radiolabel, a chromogenic label, or a fluorogenic label. Aliquots of purified receptor, receptor binding domain fragments, or cells expressing the receptor of interest on their surface are incubated with the labeled TGF-beta superfamily member in the presence of various concentrations of the unlabeled chimeric protein. The relative binding affinity of a candidate chimeric protein may be measured by quantitating the ability of the chimeric protein to inhibit the binding of the labeled TGF-beta superfamily member with the receptor. In performing the assay, fixed concentrations of the receptor and the TGF-beta superfamily member are incubated in the presence and absence of unlabeled chimeric protein. Sensitivity may be increased by preincubating the receptor with the chimeric protein before adding the labeled template TGF-beta superfamily member. After the labeled competitor has been added, sufficient time is allowed for adequate competitor binding, and then free and bound labeled TGF-beta superfamily members are separated from one another, and one or the other measured. Labels useful in the practice of the screening procedures include radioactive labels, chromogenic labels, spectroscopic labels such as those disclosed in Haughland (1994) “Handbook of Fluorescent and Research Chemicals,” 5 ed. by Molecular Probes. Inc., Eugene, Oreg., or conjugated enzymes having high turnover rates, i.e., horseradish peroxidase, alkaline phosphatase, or agalactosidase, used in combination with chemiluminescent or fluorogenic substrates. The biological activity, namely the agonist or antagonist properties of the resulting chimeric protein constructs can subsequently be characterized using conventional in vivo and in vitro assays that have been developed to measure the biological activity of any TGF-beta superfamily member. It is appreciated, however, the type of assay used preferably depends on the TGF-beta superfamily member upon which the chimeric protein is based

The presence of multimers among the subject chimeric proteins can be detected visually either by standard SDS-PAGE in the absence of a reducing agent such as DTT or by HPLC (e.g., C18 reverse phase HPLC). Multimeric proteins of an embodiment can have an apparent molecular weight proportionally greater than the monomeric subunit. e.g., in the range about 28-36 kDa for a dimer, as compared to monomeric subunits, which may have an apparent molecular weight of about 14-18 kDa. The multimeric protein can readily be visualized on an electrophoresis gel by comparison to commercially available molecular weight standards. The dimeric protein also elutes from a C18 RP HPLC (45-50% acetonitrile:0.1% TFA) at a time point different from that for its monomeric counterpart.

A second assay evaluates the presence of dimer (e.g., OP-1 dimers) by its ability to bind to hydroxyapatite. Optimally-folded dimer binds a hydroxyapatite column well in pH7, 10 mM phosphate, 6M urea, and 0.142M NaCl (dimer elutes at 0.25 M NaCl) as compared to monomer, which does not bind substantially at those concentrations (monomer elutes at 0.1 M NaCl). A third assay evaluates the presence of dimer by the protein's resistant to trypsin or pepsin digestion. The folded dimeric species is substantially resistant to both enzymes, particularly trypsin, which cleaves only a small portion of the N-terminus of the mature protein, leaving a biologically active dimeric species only slightly smaller in size than the untreated dimer (each monomer in amino acids smaller after trypsin cleavage). By contrast, the monomers and misfolded dimers are substantially degraded. In the assay, the protein is subjected to an enzyme digest using standard conditions, e.g., digestion in a standard buffer such as 50 mM Tris buffer, pH 8, containing 4 M urea, 100 mM NaCl, 0.3% Tween-80 and 20 mM methylamine. Digestion is allowed to occur at 37° C. for on the order of 16 hours, and the product visualized by any suitable means, preferably SDS PAGE.

The biological activity of the subject chimeric proteins, for example, the chimeric proteins having one or more segments from BMPs, can be assessed by any of a number of means as described in WO00/20607. For example, the protein's ability to induce endochondral bone formation can be evaluated using the well characterized rat subcutaneous bone assay. In the assay bone formation is measured by histology, as well as by alkaline phosphatase and/or osteoclacin production. In addition, osteogenic proteins having high specific bone forming activity, such as OP-1, BMP-2, BMP4, BMP-5 and BMP-6, also induce alkaline phosphatase activity in an in vitro rat osteoblast or osteosarcoma cell-based assay. Such assays are well described in the art. See, for example, Sabokdar of al. (1994) Bone and Mineral 27:57-67.; Knutsen et al. (1993) Biochem Biophvs Res. Commun 194:1352-1358; and Maliakal et al. (1994) Growth Factors 1:227-234).

By contrast, osteogenic proteins having low specific bone forming activity, such as CDMP-1 and CDMP-2, for example, do not induce alkaline phosphatase activity in the cell based osteoblast assay. For example, CDMP-1, CDMP-2 and CMDP-3 all are competent to induce bone formation, although with a lower specific activity than BMP-2, BMP-4, BMP-5, BMP-6 or OP-1. Conversely, BMP-2, BMP-4, BMP-5, BMP-6 and OP-1 all can induce articular cartilage formation, albeit with a lower specific activity than CDMP-1, CDMP-2 or CDMP-3. Accordingly, a chimeric protein having one or more segment from CDMP, designed and described herein to be a chimeric protein competent to induce alkaline phosphatase activity in the cell-based assay, is expected to demonstrate a higher specific bone forming activity in the rat animal bioassay.

The chimeric protein's biological activity can also be readily evaluated by the protein's ability to inhibit epithelial cell growth. A useful, well characterized in vitro assay utilizes mink lung cells or melanoma cells. See WO00/20607. Other assays for other members of the TGF-beta superfamily are well described in the literature and can be performed without undue experimentation.

In certain embodiment, an embodiment provides methods and agents for affecting hepatic or renal functions. Therefore, any chimeric protein of an embodiment that is expected to affect hepatic or renal function of a TGF-beta superfamily protein such as for example BMP-2, BMP-6, BMP-7, or BMP-9, can be tested in whole cells or tissues, in vitro or in vivo, to confirm their ability to modulate any functions of liver or kidney. Various methods known in the art can be utilized for this purpose.

For example, BMP-6 regulates the gene expression of hepatic hepcidin, which is the master regulator of iron homeostasis in the body. Thus, the effect of a subject chimeric protein, preferably one comprigins a segment from BMP-6, on hepcidin induction in liver can be determined by measuring mRNA or protein level of hepcidin in hepatocytes in cell based assays (see. e.g., Babitt et al., Nat. Genet. 2006, 38(5):531-9). In another example, BMP-6 and BMP-7 display anti-fibrotic properties in hepatic or renal fibrosis model. Arndt et al., Gut 64(6):973-81 (2015) discloses a BMP-6-deficient (BMP-6−/−) mouse model in which protective effects of BMP-6 on hepatic fibrosis progression has been studied. Dendooven et al., Am. J. Pathol. 178(3):1069-79 (2011) also discloses a BMP-6-deficient (BMP-6−/−) mouse model in which renal fibrosis is aggravated by unilateral ureteral obstruction, suggesting a protective role of BMP-6 against renal fibrosis. Zeisberg et al., Am. J. Physiol. Renal Physiol. 285:F1060-67 (2003) discloses two genetic mouse models in which BMP-7 inhibits progression of chronic renal fibrosis. These animal models and related in vitro or in vivo assays or methods can be utilized for evaluating the effects of a subject chimeric protein, preferably one comprising a segment from BMP-6 or BMP-7. In vitro cell based assays mentioned above may include the analysis of profibrogenic, proinflammatory, anti-fibrogenic, or anti-inflammatory gene expression in hepatic stellate cells, which represent the major source of extracellar matrix materials that causes fibrosis progression. It is understood that the screening assays of an embodiment apply to not only the subject chimeric proteins and variants thereof, but also any test compounds including agonists and antagonist of the chimeric proteins or their variants themselves. Further, these screening assays are useful for drug target verification and quality control purposes.

In other embodiment, an embodiment relates to the use of the subject chimeric TGF-beta superfamily proteins to identify compounds which can modulate activities of the chimeric proteins. Compounds identified through this screening can be tested in tissues (e.g., bone and/or cartilage) or cells (e.g., hepatic stellate cells) to assess their ability to modulate the test tissues or cells (e.g., bone/cartilage growth, or control of hepatic stellate cells) in vitro. Optionally, these compounds can further be tested in animal models to assess their ability to modulate, e.g., bone/cartilage growth or control and regulation of hepatic stellate cells in vivo.

A variety of assay formats will suffice and, in light of an embodiment, those not expressly described herein will nevertheless be comprehended by one of ordinary skill in the art. As described herein, the test compounds (agents) of an embodiment may be created by any combinatorial chemical method. Alternatively, the subject compounds may be naturally occurring biomolecules synthesized in vivo or in vitro. Compounds (agents) to be tested for their ability to act as modulators of bone or cartilage growth can be produced, for example, by bacteria, yeast, plants or other organisms (e.g., natural products), produced chemically (e.g., small molecules, including peptidomimetics), or produced recombinantly. Test compounds contemplated by an embodiment include non-peptidyl organic molecules, peptides, polypeptides, peptidomimetics, sugars, hormones, and nucleic acid molecules. In a specific embodiment, the test agent is a small organic molecule having a molecular weight of less than about 2,000 daltons.

The test compound of an embodiment can be provided as single, discrete entities, or provided in libraries of greater complexity, such as made by combinatorial chemistry. These libraries can comprise, for example, alcohols, alkyl halides, amines, amides, esters, aldehydes, ethers and other classes of organic compounds. Presentation of test compounds to the test system can be in either an isolated form or as mixtures of compounds, especially in initial screening steps. Optionally, the compounds may be optionally derivatized with other compounds and have derivatizing groups that facilitate isolation of the compounds. Non-limiting examples of derivatizing groups include biotin, fluorescein, digoxygenin, green fluorescent protein, isotopes, polyhistidine, magnetic beads, glutathione S transferase, photoactivatable crosslinkers or any combinations thereof.

In many drug screening programs which test libraries of compounds and natural extracts, high throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity or bioavailability of the test compound can be generally ignored in the in vitro system, the assay instead being focused primarily on the effect of the drug on the molecular target as may be manifest in an alteration of binding affinity between a chimeric TGF-beta superfamily protein and its binding protein (e.g., the chimeric protein itself or a TGF-beta receptor protein or fragments thereof).

Merely to illustrate, in an exemplary screening assay of an embodiment, the compound of interest is contacted with an isolated and purified chimeric protein which is ordinarily capable of binding to a TGF-beta receptor protein or fragments thereof, as appropriate for the intention of the assay. To the mixture comprising a subject chimeric protein and a TGF-beta receptor protein is then added a composition containing a test compound. Detection and quantification of the chimeric protein receptor complexes provides a means for determining the compound's efficacy at inhibiting (or potentiating) complex formation between the chimeric TGF-beta superfamily protein and its binding protein, e.g., the TGF-beta receptor or fragments thereof. The efficacy of the compound can be assessed by generating dose response curves from data obtained using various concentrations of the test compound. Moreover, a control assay can also be performed to provide a baseline for comparison. For example, in a control assay, an isolated and purified chimeric TGF-beta superfamily protein is added to a composition (cell-free or cell-based) containing a TGF-beta receptor protein or fragment thereof, and the formation of the chimeric protein-receptor complex is quantitated in the absence of the test compound. It will be understood that, in general, the order in which the reactants may be admixed can be varied, and can be admixed simultaneously. Moreover, in place of purified proteins, cellular extracts and lysates may be used to render a suitable cell-free assay system. Alternatively, cells expressing a TGF-beta receptor protein or fragments thereof on their surfaces can be used in certain assays.

Complex formation between a subject chimeric TGF-beta superfamily protein and its binding protein may be detected by a variety of techniques. For instance, modulation of the formation of complexes can be quantitated using, for example, detectably labeled proteins such as radiolabelled (e.g., 32P, 35S, 14C or 3H), fluorescently labeled (e.g., FITC), or enzymatically labeled chimeric protein or its binding protein, by immunoassay, or by chromatographic detection.

An embodiment contemplates the use of fluorescence polarization assays and fluorescence resonance energy transfer (FRET) assays in measuring, either directly or indirectly, the degree of interaction between a chimeric TGF-beta superfamily protein and its binding protein (e.g., a TGF-beta receptor protein or fragments thereof). Further, other modes of detection such as those based on optical waveguides (PCT Publication WO 96/26432 and U.S. Pat. No. 5,677,196), surface plasmon resonance (SPR), surface charge sensors, and surface force sensors are compatible with many embodiments of the present invention.

Moreover, an embodiment contemplates the use of an interaction trap assay, also known as the “two hybrid assay,” for identifying agents that disrupt or potentiate interaction between a chimeric TGF-beta superfamily protein and its binding protein (e.g., a TGF-beta receptor protein or fragments thereof). See for example, U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696).

A chimera polynucleotide, a polypeptide, an antibody, a cell and other reagent of an embodiment have a wide variety of uses, both in vitro and in vivo. For example, in representative embodiments, these reagents may be used in vitro or in vivo (e.g., in an animal model) to study the processes of mineralization, bone formation, and bone loss. Further. “knock in” and “knock out” animals can be used as animal models of disease or as screening tools (discussed more below) for compounds that interact with the chimera polynucleotide or polypeptide. It will be apparent to those skilled in the art that any suitable vector can be used to deliver the polynucleotide to a cell or subject. The choice of delivery vector can be made based on a number of factors known in the art, including age and species of the target host, in vitro versus in vivo delivery, level and persistence of expression desired, intended purpose (e.g., for therapy or screening), the target cell or organ, route of delivery, size of the isolated polynucleotide, safety concerns, and the like.

Chimeric polypeptide of an embodiment may be formulated for use in various biological systems including in vivo. Any of a variety of art-known methods can be used to administer a chimera either alone or in combination with other active agents. For example, administration can be parenterally by injection or by gradual infusion over time. The agent (s) can be administered by such means as oral, rectal, buccal (e.g., sublingual), vaginal, parenteral (e.g., subcutaneous, intramuscular including skeletal muscle, cardiac muscle, diaphragm muscle and smooth muscle, intradermal, intravenous, intraperitoneal), topical (i.e., both skin and mucosal surfaces, including airway surfaces), intranasal, transdermal, intraarticular, intrathecal, intracavity, and inhalation administration, administration to the liver by intraportal delivery, as well as direct organ injection (e.g., into the liver, into the brain for delivery to the central nervous system, into the pancreas). The most suitable route in any given case will depend on the nature and severity of the condition being treated and on the nature of the particular compound which is being used.

An embodiment also provides a pharmaceutical preparation comprising a subject chimeric protein and a pharmaceutically acceptable carrier. A pharmaceutical preparation may be employed to promote growth of a tissue or diminishing or prevent loss of a tissue in a subject, preferably a human. The targeted tissue can be, for example, bone, cartilage, skeletal muscle, cardiac muscle and/or neuronal tissue.

In another aspect, a chimeric TGF-beta polypeptide can be formulated either alone or in combination with other agents for administration (e.g., as a lotion, cream, spray, gel, or ointment). It may be formulated into liposomes to reduce toxicity or increase bioavailability. Other methods for delivery include oral methods that entail encapsulation of the in microspheres or proteinoids, aerosol delivery (e.g., to the lungs), or transdermal delivery (e.g., by iontophoresis or transdermal electroporation). Other methods of administration will be known to those skilled in the art.

Preparations for parenteral administration of a composition comprising a chimeric TGF-beta polypeptide include sterile aqueous or non-aqueous solutions, suspensions, and emulsions. Examples of non-aqueous solvents are propylene glycol, polyethylene glycol, vegetable oils (e.g., olive oil), and injectable organic esters such as ethyl oleate. Examples of aqueous carriers include water, saline, and buffered media, alcoholic/aqueous solutions, and emulsions or suspensions. Examples of parenteral vehicles include sodium chloride solution, Ringer's dextrose, dextrose and sodium chloride, lactated Ringer's, and fixed oils. Intravenous vehicles include fluid and nutrient replenishers, electrolyte replenishers (such as those based on Ringer's dextrose), and the like. Preservatives and other additives such as, other antimicrobial, anti-oxidants, cheating agents, inert gases and the like also can be included.

An embodiment provides various disease and disorders that may be modulated by a TGF-beta protein family member comprising contacting or administering a therapeutically effective amount of a chimeric TGF-beta polypeptide either alone or in combination with other agents to a subject who has, or is at risk of having, such a disorder.

A therapeutically effective amount can be measured as the amount sufficient to decrease a subject's symptoms associated with the diseases or disorder. Typically, the subject is treated with an amount of a therapeutic composition sufficient to reduce a symptom of a disease or disorder by at least 50%, 90% or 100%. Generally, the optimal dosage will depend upon the disorder and factors such as the weight of the subject, the age, the weight, sex, and degree of symptoms. For example, with respect to bone morphogenesis, optionally, the dosage may vary with the type of matrix used in the reconstitution and the types of compounds in the composition. The addition of other known growth factors to the final composition, may also affect the dosage. Progress can be monitored by periodic assessment of bone growth and/or repair, for example, X-rays, histomorphometric determinations, and tetracycline labeling. Nonetheless, suitable dosages can readily be determined by one skilled in the art. Typically, a suitable dosage is 0.01 to 40 mg/kg body weight.

As mentioned previously, the composition and the method of an embodiment can include the use of additional (e.g., in addition to a chimeric TGF-beta polypeptide) therapeutic agents (e.g., an inhibitor of TNF, an antibiotic, and the like). The chimeric TGF-beta polypeptide, other therapeutic agent(s), and/or antibiotic(s) can be administered, simultaneously, but may also be administered sequentially.

A pharmaceutical composition comprising a chimera according to an embodiment can be in a form suitable for administration to a subject using carriers, excipients, and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol, and polyhydric alcohols. Intravenous vehicles include fluid and nutrient replenishers. Preservatives include antimicrobial, anti-oxidants, chelating agents, and inert gases. Other pharmaceutically acceptable carriers include aqueous solutions, non-toxic excipients, including salts, preservatives, buffers and the like, as described, for instance, in Remington's Pharmaceutical Sciences, 15th ed., Easton: Mack Publishing Co., 1405-1412, 1461-1487 (1975), and The National Formulary XIV., 14th ed., Washington: American Pharmaceutical Association (1975), the contents of which are hereby incorporated by reference. The pH and exact concentration of the various components of the pharmaceutical composition are adjusted according to routine skills in the art. See Goodman and Gilman's, The Pharmacological Basis for Therapeutics (7th ed.).

The pharmaceutical composition according to an embodiment may be administered locally or systemically. A “therapeutically effective dose” is the quantity of an agent according to an embodiment necessary to prevent, to cure, or at least partially arrest a symptoms associated with a disease or disorder or to promote cell growth, proliferation or differentiation. Amounts effective for this use will, of course, depend on the severity of the disease, disorder, or desired effect and will depend on weight and general state of the subject. Typically, dosages used in vitro may provide useful guidance in the amounts useful for in situ administration of the pharmaceutical composition, and animal models may be used to determine effective dosages for treatment of the disease or disorder. Various considerations are described, e.g., in Langer, Science, 249: 1527, (1990); Gilman et al. (eds.) (1990), each of which is herein incorporated by reference. Dosages of pharmaceutically active compounds can be determined by methods known in the art, see, e.g., Remington's Pharmaceutical Sciences (Maack Publishing Co., Easton. Pa.); Remington, The Science & Practice of Pharmacy, (Lippincott Williams & Wilkins; Twenty first Edition). The therapeutically effective dosage of any specific compound will vary somewhat from compound to compound, and patient to patient, and will depend upon the condition of the patient and the route of delivery. As a general proposition, a dosage from about 0.1 to about 100 mg/kg will have therapeutic efficacy, with all weights being calculated based upon the weight of the compound, including the cases where a salt is employed. Toxicity concerns at the higher level can restrict intravenous dosages to a lower level such as up to about 10 to about 20 mg/kg, with all weights being calculated based upon the weight of the compound, including the cases where a salt is employed. A dosage from about 10 mg/kg to about 50 mg/kg can be employed for oral administration. Typically, a dosage from about 0.5 mg/kg to 15 mg/kg can be employed for intramuscular injection. Particular dosages are about 1 μmol/kg to 50 μmol/kg, and more particularly to about 22 μmol/kg and to 33 μmol/kg of the compound for intravenous or oral administration, respectively.

In an embodiment, more than one administration (e.g., two, three, four, or more administrations) can be employed over a variety of time intervals (e.g., hourly, daily, weekly, monthly, etc.) to achieve therapeutic effects.

The composition and chimera of an embodiment find use in veterinary and medical applications. Suitable subjects include both avians and mammals, with mammals being preferred. The term “avian” as used herein includes, but is not limited to, chickens, ducks, geese, quail, turkeys, and pheasants. The term “mammal” as used herein includes, but is not limited to, humans, bovines, ovines, caprines, equines, felines, canines, lagomorphs, etc. Human subjects include neonates, infants, juveniles, and adults. In other embodiments, the subject is an animal model of a liver disease or a bone and cartilage disease.

As used herein, “administering a therapeutically effective amount” is intended to include methods of giving or applying a pharmaceutical composition of an embodiment to a subject that allow the composition to perform its intended therapeutic function.

The pharmaceutical composition can be administered in a convenient manner, such as by injection (subcutaneous, intravenous, etc.), oral administration, inhalation, transdermal application, or rectal administration. Depending on the route of administration, the pharmaceutical composition can be coated with a material to protect the pharmaceutical composition from the action of enzymes, acids, and other natural conditions that may inactivate the pharmaceutical composition. The pharmaceutical composition can also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersions. In all cases, the composition should be sterile and should be fluid to the extent that easy syringability exists. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size, in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be typical to include isotonic agents, for example, sugars, polyalcohols, such as mannitol, sorbitol, or sodium chloride in the composition. Prolonged absorption of the injectable composition can be brought about by including in the composition an agent that delays absorption, for example, aluminum monostearate and gelatin.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the pharmaceutical composition into a sterile vehicle that contains a basic dispersion medium and the required other ingredients from those enumerated above.

The pharmaceutical composition can be orally administered, for example, with an inert diluent or an assimilable edible carrier. The pharmaceutical composition and other ingredients can also be enclosed in a hard or soft-shell gelatin capsule, compressed into tablets, or incorporated directly into the individual's diet. For oral therapeutic administration, the pharmaceutical composition can be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations can, of course, be varied and can conveniently be between about 5% to about 80% of the weight of the unit.

The tablets, troches, pills, capsules, and the like can also contain the following: a binder, such as gum gragacanth, acacia, corn starch, or gelatin; excipients such as dicalcium phosphate; a disintegrating agent, such as corn starch, potato starch, alginic acid, and the like; a lubricant, such as magnesium stearate; and a sweetening agent, such as sucrose, lactose or saccharin, or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar, or both. A syrup or elixir can contain the agent, sucrose as a sweetening agent, methyl and propylparabens as preservatives, a dye, and flavoring, such as cherry or orange flavor. Of course, any material used in preparing any dosage unit form should be pharmaceutically pure and substantially non-toxic/biocompatible in the amounts employed. In addition, the pharmaceutical composition can be incorporated into sustained-release preparations and formulations.

Thus, a “pharmaceutically acceptable carrier” is intended to include solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the pharmaceutical composition, use thereof in the therapeutic composition or the method of treatment is contemplated. Supplementary active compounds can also be incorporated into the composition.

In certain embodiments, the therapeutic method includes administering the composition topically, systemically, or locally as an implant or device. When administered, the therapeutic composition described by an embodiment is generally in a pyrogen-free, physiologically acceptable form. Further, the composition may desirably be encapsulated or injected in a viscous form for delivery to the site of bone, cartilage or tissue damage. Topical administration may be suitable for wound healing and tissue repair. Therapeutically useful agents other than the chimera of an embodiment may also optionally be included in the composition as described above, may alternatively or additionally, be administered simultaneously or sequentially with the chimera in the methods of the described herein. For example, preferably for bone and/or cartilage formation, the composition would include a matrix capable of delivering a BMP chimera or other therapeutic compounds to the site of bone and/or cartilage damage, providing a structure for the developing bone and cartilage and optimally capable of being resorbed into the body. For example, the matrix may provide slow release of the BMP chimera. Such matrices may be formed of materials presently in use for other implanted medical applications.

The choice of matrix material is based on biocompatibility, biodegradability, mechanical properties, cosmetic appearance and interface properties. The particular application of the subject composition will define the appropriate formulation. Potential matrices for the composition may be biodegradable and chemically defined calcium sulfate, tricalciumphosphate, hydroxyapatite, polylactic acid and polyanhydrides. Other potential materials are biodegradable and biologically well defined, such as bone or dermal collagen. Further matrices are comprised of pure proteins or extracellular matrix components. Other potential matrices are non-biodegradable and chemically defined, such as sintered hydroxyapatite, bioglass, aluminates, or other ceramics. Matrices may be comprised of combinations of any of the aforementioned types of material, such as polylactic acid and hydroxyapatite or collagen and tricalciumphosphate. The bioceramics may be altered in composition, such as in calcium-aluminate-phosphate and processing to alter pore size, particle size, particle shape, and biodegradability.

Certain compositions disclosed herein may be administered topically, either to skin or to mucosal membranes. The topical formulations may further include one or more of the wide variety of agents known to be effective as skin or stratum corneum penetration enhancers. Examples of these are 2-pyrrolidone, N-methyl-2-pyrrolidone, dimethylacetamide, dimethylformamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface active agents. Keratolytic agents such as those known in the art may also be included. Examples are salicylic acid and sulfur.

It is especially advantageous to formulate a parenteral composition in dosage unit form for ease of administration and uniformity of dosage. “Dosage unit form” as used herein, refers to physically discrete units suited as unitary dosages for the individual to be treated; each unit containing a predetermined quantity of pharmaceutical composition is calculated to produce the desired therapeutic effect in association with the required pharmaceutical carrier. The specification for the dosage unit forms of an embodiment are related to the characteristics of the pharmaceutical composition and the particular therapeutic effect to be achieve.

The principal pharmaceutical composition is compounded for convenient and effective administration in effective amounts with a suitable pharmaceutically acceptable carrier in an acceptable dosage unit. In the case of the composition containing supplementary active ingredients, the dosage is determined by reference to the usual dose and manner of administration of the said ingredients.

One of the challenges to using a chimera as therapeutics is the ability to deliver the proteins effectively. The chimera of an embodiment can be delivered by several different methods. In the blood stream, the half-life of most TGF-β ligands is on the order of minutes. To compensate for the ligands being degraded so quickly, current therapies involving TGF-β ligands use very high doses of the proteins. Alternatively, several means to directly modify the ligands or delivery systems are available to help improve the stability or sustained release properties of the ligands.

(1) Direct modification of the protein includes PEGylation as one common form of modification. In this method, polyethylene glycol (PEG) is covalently attached to the protein in hopes of improving stability by increasing solubility, resistance to proteolysis, and decreased immunogenicity.

(2) Rational modification of residues on the protein surface. By improving any electrostatic instability, without changing overall protein function, the overall stability of molecule can be improved. Using continuum electrostatic models, residues contributing to instability can be located and then analyzed to see if it can be mutated to a more favorable residue.

(3) Fusing the ligand to another protein or portion of a protein is another technique to increase protein stability and solubility. The antibody constant fragment (Fc) is common fusion partner used to improve the stability and solubility.

(4) The use of liposomes can be used as a protein delivery vehicle. Liposomes are composed of any number of different phospholipids, which self-assemble to form spheres. The protein of interest is encapsulated inside the bilayer, protecting it from the outside environment. The phospholipid composition influences the exact properties of the liposome and can be tailored to release the protein under any number of desired conditions. Polymer/liposome composite systems are also available to be used as delivery systems. Ideally, this type of system combines the advantages of each system to improve protein delivery.

(5) Similar to liposomes, polymers can be used as protein drug delivery systems. The polymers are used to make a matrix, commonly what is termed a hydrogel due to the high water content of the material. The advantage of using the gel is it allows for long term, sustained release as well as protecting the protein from proteolysis. As with the liposomes, the polymers used to make the gel influence its properties. There are two general classifications for the materials used to make the hydorgels: natural and unnatural polymers. Common materials used to create hydrogels using natural polymers include collagen, gelatin, fibrin, Hyaluronic acid, alginate, chitosan, and dextran. Synthetic polymers used to make hydrogels include Poly(ethylene oxide). Poly(acrylic acid), Poly(N-isopropylacrylamide), Poly(vinyl alcohol), and Polyphosphazene.

(6) A different kind of hydrogel can be created without the use of polymers, either natural or unnatural. Considered to be a bioactive glass, or Xerogel, this material is created from silica and calcium phosphate layer capable of absorbing the protein of interest. The Xerogel increases the sustained release time of the protein up to weeks. Results from cell viability assay using osteoblast cell line MC3T3 by MTT assay show that the xerogel material is nontoxic up to the highest concentration of 30 mg/ml in the culture media we tested.

The chimeric polypeptide of an embodiment alone or in combination can be used to treat any number of liver disease or disorders where modulating of TGF-beta activity provides a therapeutic benefit. For example, the chimera of an embodiment can be used in subjects suffering from a liver disease, including, but not limited to, non-alcoholic fatty liver disease, liver fibrosis and hepatic inflammation.

### EXAMPLES

### Example 1

Description of Subdomains (Building Blocks) for Generating Designer Ligand

In order to create the chimeras, a first step was deciding where to make the borders for each of the segments. The chimera library has been constructed using activin-βA and BMP-6 as two sequence sources. To design the cut-off regions (Junction) for the sections to make the activin/BMP-6 (AB) chimera, a structure-guided approach combined with protein sequence alignment was used. Initially, the 3-dimensional crystal structures of activin-βA (Harrington et al., 2006) and BMP-6 (Allendorph et al., 2007) were inspected structurally. From this analysis, the ligands were loosely divided into 6 distinct sections (see FIG. 4 for segments 1 through 6). The exact segment junctions were ultimately determined following a protein sequence alignment of the two ligands to minimize any sequence changes of either protein sequence as a result of joining the Junction. Further, the segmental boundaries were chosen to be located in structural regions away from receptor binding sites.

Detailed descriptions of Junctions: Between segments 1 and 2 (Junction 1): Focusing on the boundary of segment 1 and segment 2, we found a 10-residue region that is highly conserved between BMP-6 and activin-βA. Indeed, 8 of the 10 residues are identical and the other two are very conservative differences. This area is located in the tip region of Finger 1 and depending of the ligand, makes or is predicted to make limited contacts with either receptor type. Based on the ternary crystal structure of BMP-2/BMPRIa/ActRII (Allendorph et al., 2006), only Val-26, Gly-27, and Trp-28 (BMP-2 numbering) generate contacts with the type I receptor. Of these three residues, only Val-26 is different between the ligands, but it is a, very conservative change since the corresponding residue in activin-βA and BMP-6 is Ile-23 and Leu-43, respectively. Since the residues in this region are very similar and not involved in receptor binding, it makes for a good boundary point for segment 1 and 2.

Between segments 2 and 3 (Junction 2): Moving to the boundary region between segments 2 and 3, another good area for our boundary cut-off can be found. Here exists a highly conserved region of 6 residues between activin-βA and BMP-6, where 4 of the 6 residues are identical. When the ligands are properly folded, this region is located in the center of the dimer, with both cyteines participating in the cystine knot. This is advantageous because the residues here are buried from the surface of the ligands and do not participate in any ligand-receptor interactions.

Between segments 4 and 5 (Junction 4): Similar to the segment 2/3 boundary, the segment 4/5 boundary is situated in an excellent location for the cut-off. Here, we find a 6-residue region that is highly conserved between activin-βA and BMP-6 with 4 identical residues out of the 6 residues, and this region is also buried at the center of the ligand dimer. The 2 cysteine residues participate in both the cystine knot as well as the inter-monomer disulfide bond. Again, this location prevents the residues in this region from participating in receptor binding interactions.

Between segments 5 and 6 (Junction 5): To extend the design of BMP-6 and activin-βA chimeras, other boundary regions have been chosen to facilitate generating RASCH constructs using all members of the TGF-β superfamily. Along with sharing structural architecture, the TGF-β superfamily ligands seem to have certain regions in their protein sequences that are highly conserved. Interestingly, these regions coincide with the boundary regions chosen for making the BMP-2 and activin-βA chimeras. For example, in the boundary region of 4 and 5, most ligands share 3 out of the 4 residues that define the boundary domain. This high degree of similarity, coupled with these regions being isolated from the receptor binding sites, indicates RASCH as the universal strategy to create a library of Designer Ligands with new functionalities.

Between segments 3 and 4 ((Junction 3): The boundary between segments 3 and 4 is subject to structural variability between different subfamilies, in which ligand-receptor assembly mechanism can differ substantially. In that regard, segments 3 and 4 can be treated as one segmental piece such that two segments will be derived from the common parental strand to preserve their structural integrity.

The structural similarity among all TGF-beta superfamily ligands forms the rational basis for designing chimeric protein by exchanging (swapping) related segments of the sequences known to carry out certain functionality such as molecular recognition. Protein engineering of Antibody chain, or more specifically of antibody fragment (Fab), will be a prime example where the basic structural scaffold is built on the Core architecture of the light- and heavy chain sequences, for which six variable loops, three from each of the two chains, are responsible for the role of epitope-binding specificity. In the similar vein, the TGF-beta superfamily ligands share their structural framework as a butterfly-like architecture. A portion(s) of the sequence segments functionally equivalent to variable loop regions of Antibody can then be ‘implanted’ to transfer recognition specificity from one ligand to another. Our design principle distinguishes itself from the aforementioned ‘functional transfer by sequence implantation’. The new chimeric library is created on the basis of structural feasibility of each subdomain as defined by each Junction. The junctions between the various domains of the TGF-beta family members used to generate the chimeras of the disclosure provide useful building blocks of the chimera library. By this reasoning, Junctions 1, 2, 4, and 5 are well defined to be broadly applicable to all TGF-beta superfamily members, whereas Junction 3 is not broadly applicable. The application of Junction 3 in the chimera design depends on the target sequences, in which case subdomain segments 3 and 4 can be treated instead as one segment in designing the chimera library. The approach maximizes the chance of producing such protein products that are foldable, for which functional characterization will then follow.

### Example 2

Generation of TGF-β Chimeras

To generate these novel TGF-β ligands, a modified directed evolution approach was utilized. Typically, this technique involves making a large number of random protein sequences, greater than 10³, either by mixing the sequences of homologous genes or inserting random mutations and then screening for the desired ligand properties. Using a structure guided approach, several TGF-β ligand crystal structures were analyzed and divided into 6 distinct sections. These sections roughly encompass the following regions of the ligand: section 1, N-terminus and beta strand 1; section 2, beta strand 2; section 3, pre-helix loop; section 4, alpha helix; section 5, beta strand 3; and section 6, beta stand 4 and C-terminus. Using this protocol, 64 different ligand combinations are possible for each set of TGF-β ligands chosen to be recombined. When two or more parental chains are from different subfamilies (e.g. BMP/GDF v.s. TGFbeta), the difference between their signaling mechanisms may not be captured if sections 3 and 4 are separated. To be broadly applicable as the design principle, it is also part of the design to keep two structural segments, sections 3 and 4, can be treated as one section of either of the parental gene (referred to as section 3*4).

The strategy was implemented by making activin/BMP-6 chimeras, where Activin-βA was picked as the target ligand as it has high affinity to TGF-β type II receptors. BMP-6 was chosen as it is biologically very interesting. To design the various sections, a sequence alignment of BMP-6 and activin-βA was performed to locate regions of sequence identity between the ligands (FIG. 4). These regions were used as the boundaries for the different sections. By using these parts of the sequence as the overlap regions for the oligonucleotides during PCR, changes will not be introduced into either the BMP-6 or activin-βA sequences. The sequence alignment was then used in conjunction with data from previously solved BMP-6 and activin-βA structures to ultimately determine the 6 sections (FIG. 4 a-c). Due to limitations with regions of identity between the sequences, the sections had to be shifted slightly from ideal. Particularly, the pre-helix loop and the majority of the α-helix were combined into one section, while the remainder of the α-helix to the beginning of beta strand 3 was placed into a different section (FIG. 4 b and c).

The chimeras were labeled according to the sections they contained. For example, 1b2b3b4a5a6b, in which the b's represent that the section was taken from BMP-6 and the a's represent that the section was derived from activin-βA.

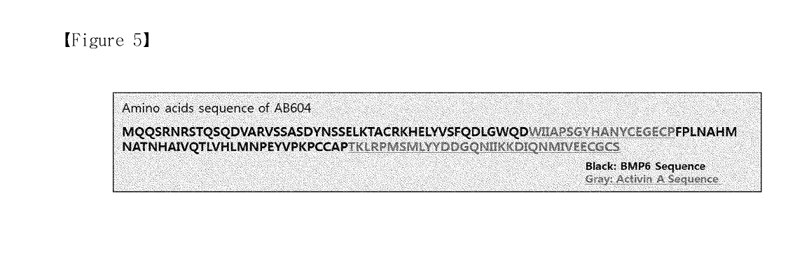

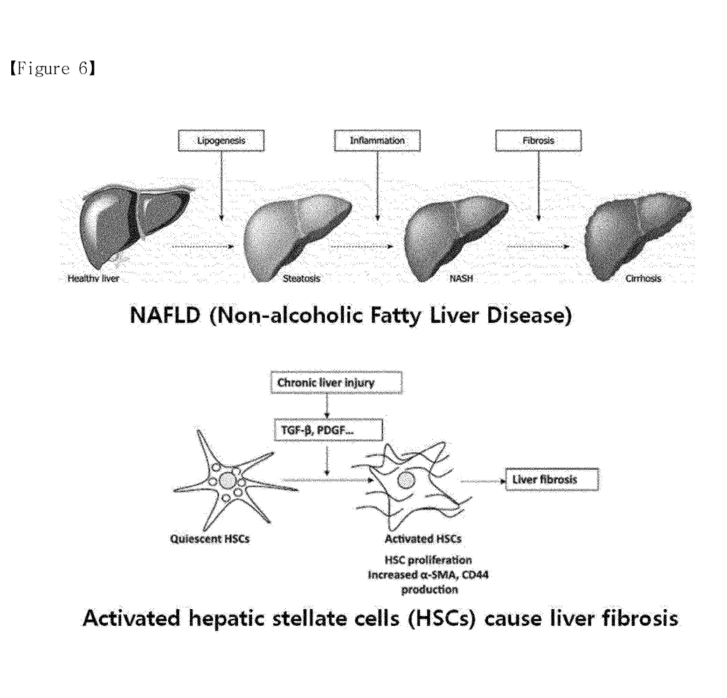

One of the activin/BMP-6 chimeras, designated as AB604 is shown in FIG. 5.

### Example 3

Activin/BMP-6 Chimeric Proteins Suppress Liver Fibrosis

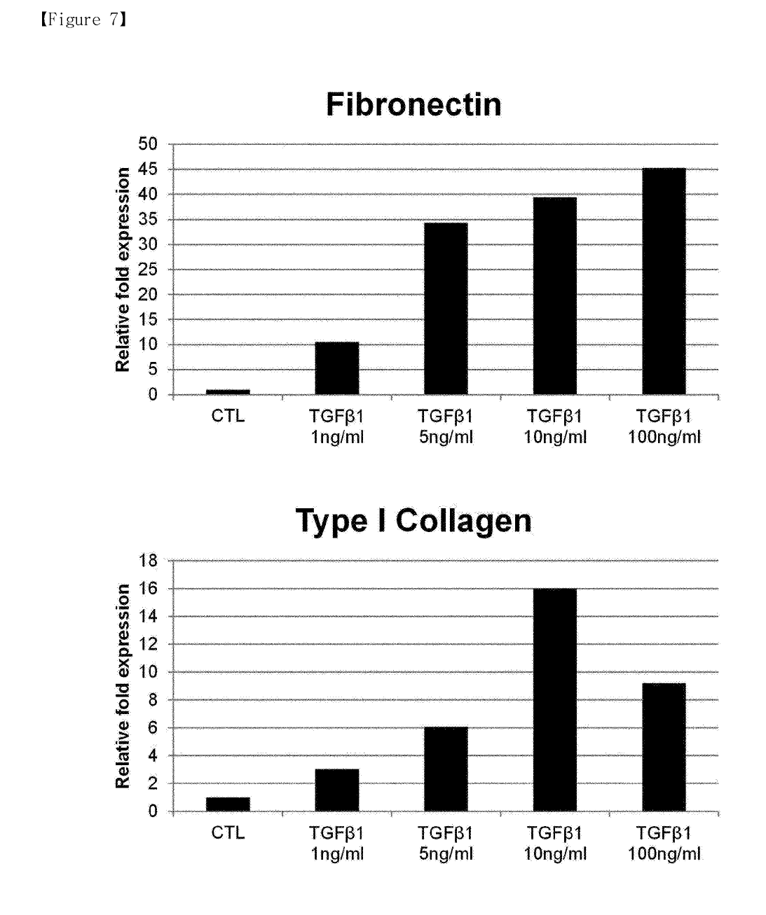

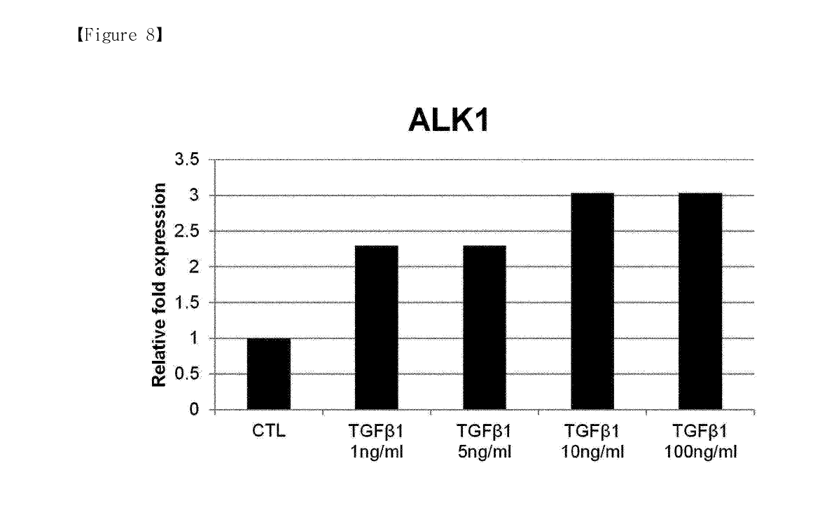

To test the effects of TGFβ1 on liver fibrosis induction, LX-2 cells were treated with 1.0 ng/mL, 5.0 ng/mL, 10 ng/mL, or 100 ng/mL of TGFβ1 and expression levels of marker genes were measured using real-time pPCR. The expression of fibrosis marker genes such as Fibronectin, type I Collagen, ALK1 were elevated by treatment of TGFβ1 in a dose dependent manner (FIGS. 7 and 8).

To test the effects of activin/BMP-6 chimeric proteins on suppressing liver fibrosis, LX-2 cells were first treated with 1 ng/mL of TGFβ1. After the TGFβ1 treatment, the LX-2 cells were treated with 10 ng/mL, 100 ng/mL, or 200 ng/mL of AB604, BMP-2, or BMP-6, for 12 hrs of 24 hrs. Then gene expression levels of α-SMA, Collagen1 (COL1), or Fibronectin were measured using real-time pPCR. Vehicle control (untreated) and positive control (treated with 1 ng/nL TGFβ1) were used for comparison.

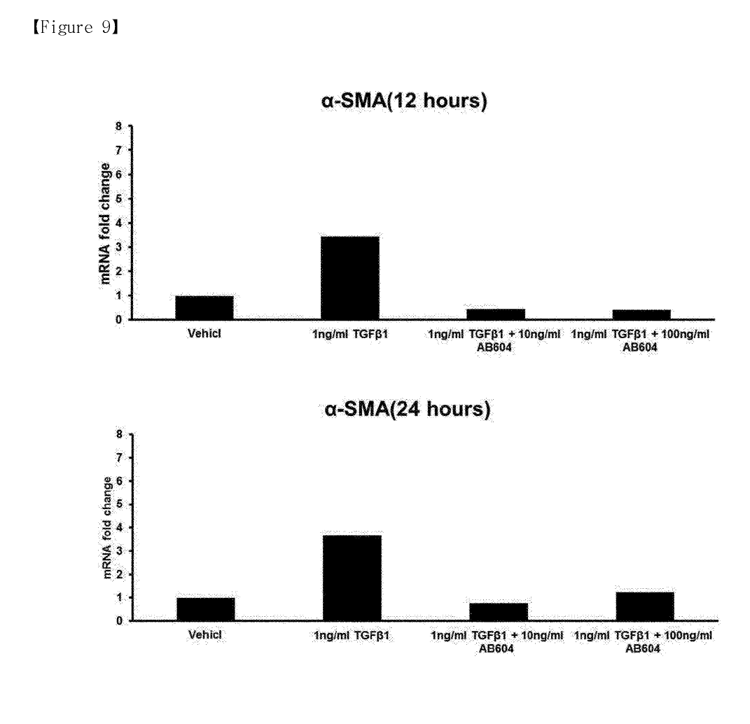

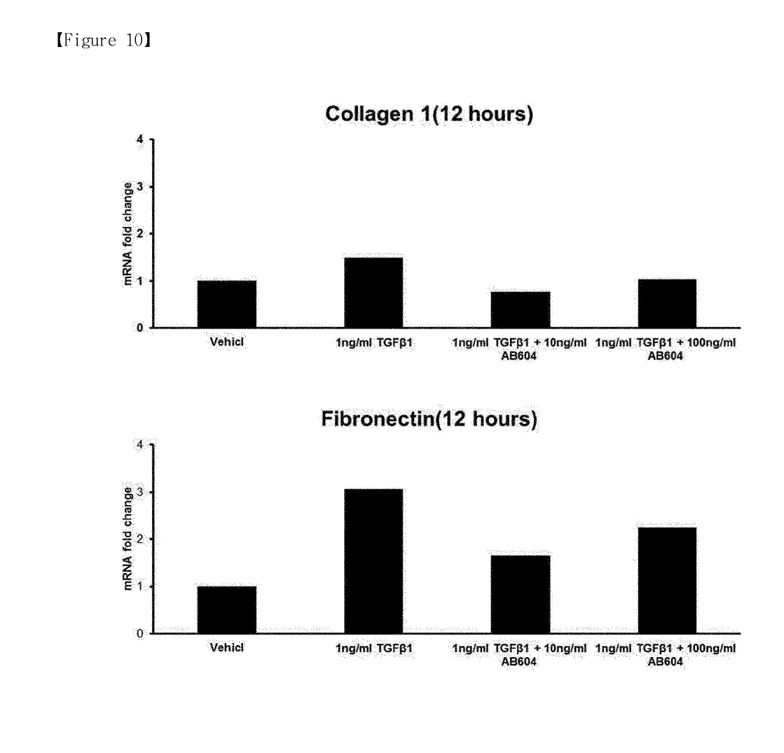

AB604 effectively suppressed the TGFβ1-induced α-SMA gene expression (FIG. 9), and TGFβ1-induced Collagen1 (COL1) and Fibronectin gene expression (FIG. 10).

### Example 4

Inhibition of TGFβ Signaling

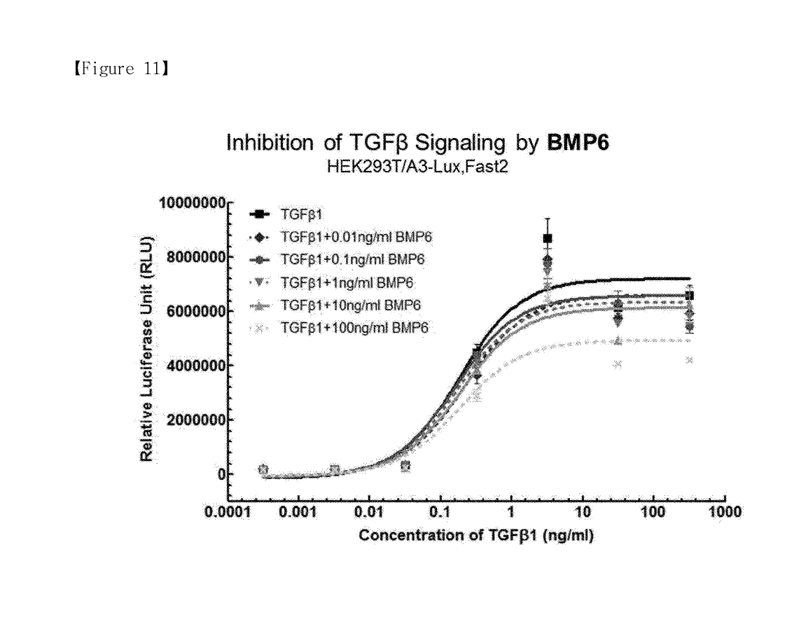

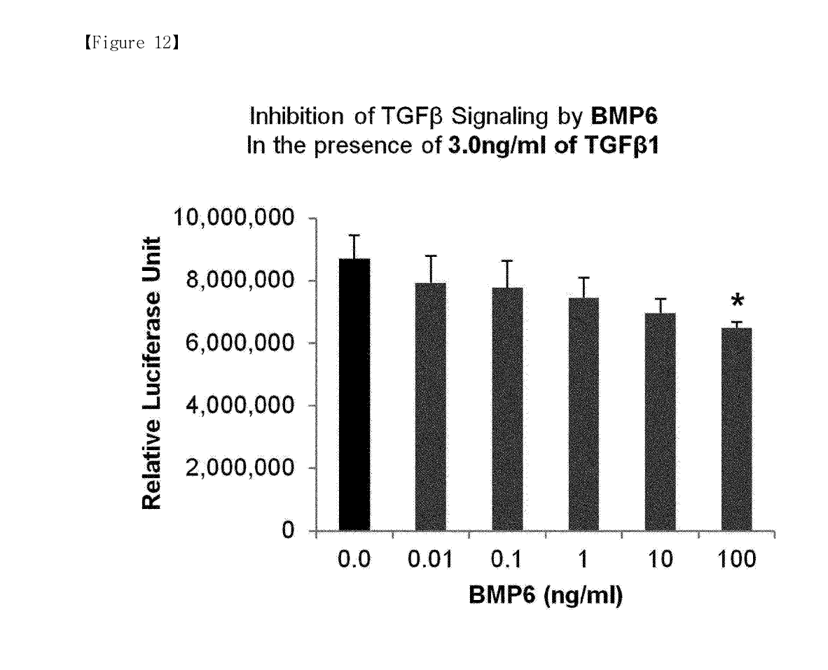

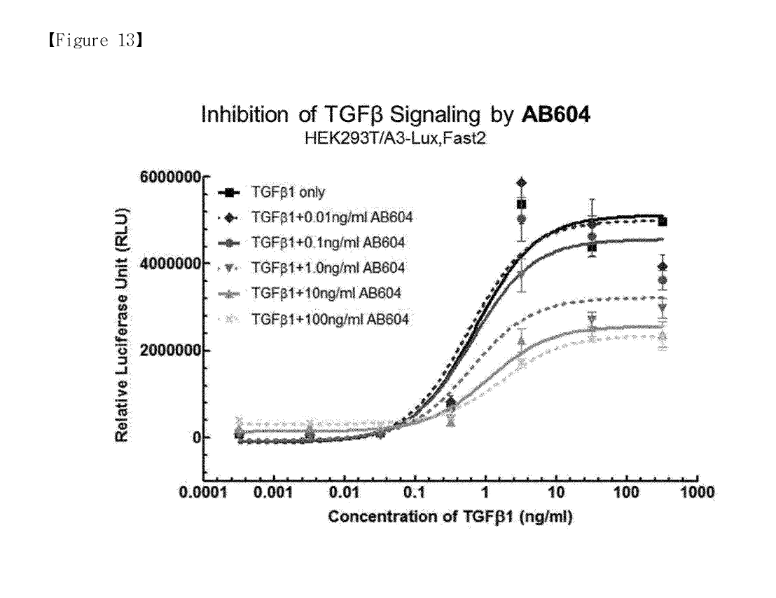

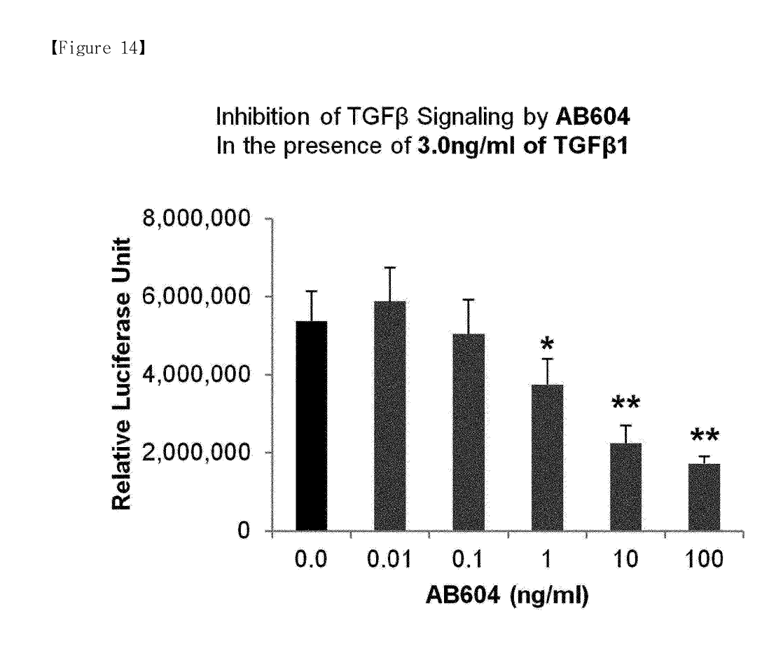

AB604 was shown to inhibit the signaling activities of TGFβ1 more potently than BMP6 in HEK293T cells as observed by a luciferase reporter assay. When TGFβ1-responsive A3 promoter was activated by TGFβ1 in HEK293T cells in the presence or absence of BMP6 or AB604 at various concentrations, the maximally attainable signaling activity by TGFβ1 was suppressed by the presence of BMP6 or AB604 in a dose-dependent manner (FIG. 11 to FIG. 14). This suppressive effect was more prominent by AB604 than by BMP6. For example, FIG. 12 and FIG. 14 show dose-dependent decreases of TGFβ1-mediated luciferase activities by BMP6 and AB604, respectively. AB604 displayed a statistically significant reduction of TGFβ1 activities already at 1 ng/ml (FIG. 12), whereas BMP6 did so at 100 ng/ml (FIG. 14)

### Example 5

Activation of Hepcidin Gene Expression

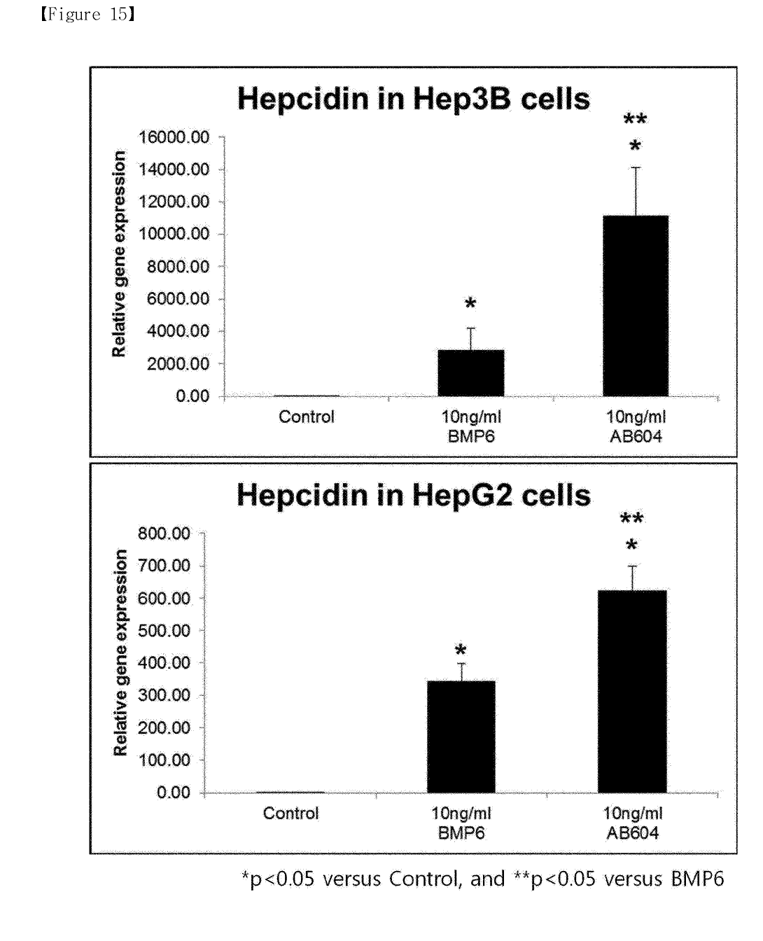

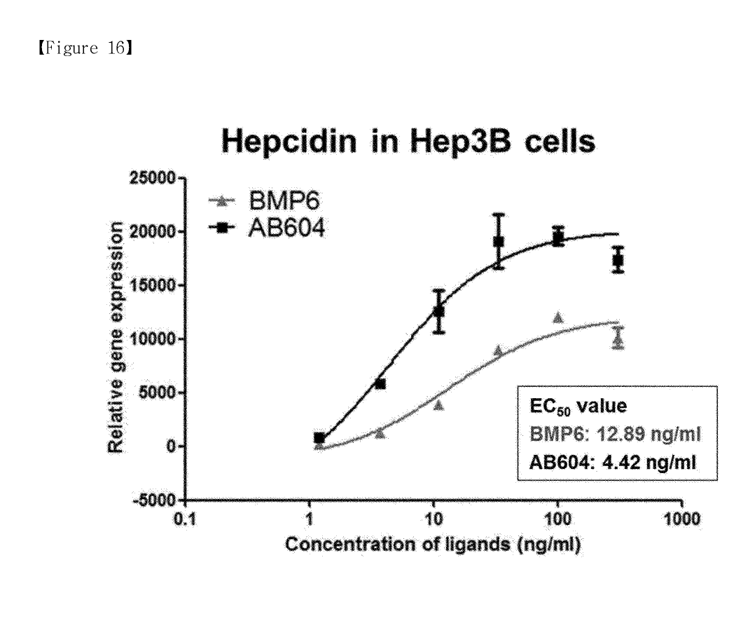

BMP6 regulates the expression of hepcidin in hepatocytes. Hepcidin is known as a master regulator of systemic and hepatic iron homeostasis, by its action of degrading ferroportin (FPN), an iron-efflux transporter in cell membranes. The effects of BMP6 and AB604 in inducing hepcidin gene expression were tested in Hep3B and HepG2 cells. Both BMP6 and AB604 have increased mRNA level of hepcidin significantly in both Hep3B cells and HepG2 cells, compared to the control. Moreover, the increase of mRNA level of hepcidin was significantly greater in AB604-treated cells than BMP6-treated counterparts (FIG. 15). The dose-dependent profile of hepcidin gene expression demonstrated the higher potency of AB604 compared to that of BMP6, where EC50 values of BMP6 and AB604 exhibited 3-fold differences (12.89 ng/ml for BMP6, 4.42 ng/ml for AB604) (FIG. 16).

### Example 6

Inhibition of Fibronectin Expression

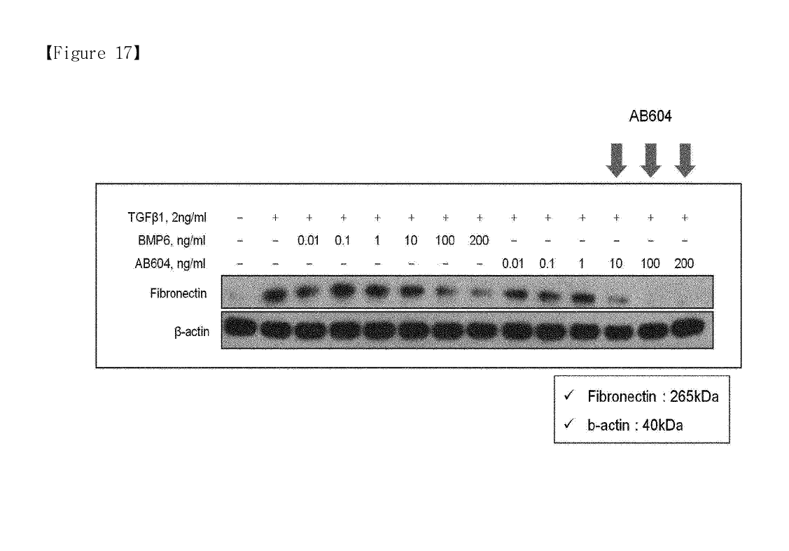

Fibronectin is one of the extracellular matrix proteins that are expressed in hepatic stellate cells (HSC) upon HSC activation by TGFβ1. To see if AB604 or BMP6 can suppress the protein expression of fibronectin in HSCs, LX-2 cells (human hepatic stellate cell line) were activated with 2 ng/ml of TGFβ1 and also treated with BMP6 or AB604 at the indicated concentrations (0.01 ng/ml, 0.1 ng/ml, 1 ng/ml, 10 ng/ml, 100 ng/ml, and 200 ng/ml) (FIG. 17). TGFβ1 induced expression of fibronectin (lane 2 in FIG. 17) and both BMP6 and AB604 suppressed TGFβ1-mediated fibronectin production in a dose dependent manner. The suppressive effect of AB604 was superior to BMP6, for AB604 showed significant suppression already at 10 ng/ml compared to the equivalent suppression at 200 ng/ml of BMP6.

### Example 7

Suppression of CDAA-Diet Induced Increase in Liver Weight

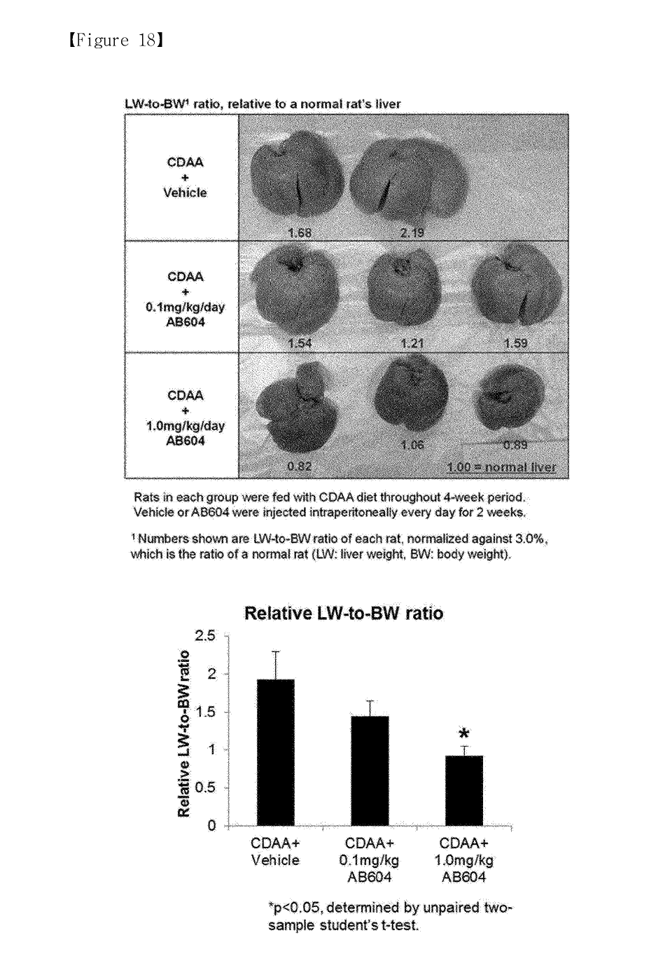

Choline-deficient L-amino acid-defined (CDAA) diet results in rapid progression of liver steatosis followed by a rise in ALT in rats, and long period of CDAA diet leads to NASH (non-alcoholic steatohepatitis), liver fibrosis, and liver cancer. Male Wistar rats were fed with CDAA for 2 weeks and AB604 was intraperitoneally injected (0.1 mg/kg/day or 1.0 mg/kg/day) for two additional weeks with continued CDAA diet. When rats were sacrificed at the 4^{th} week, the relative liver weights were compared using the ratio of liver weight to body weight (LW-to-BW ratio). The pictures of whole livers in FIG. 18 show a tendency of liver size decrease by AB604. Numbers next to the livers show LW-to-BW ratio of each rat, normalized against the corresponding ratio of normal rat, 3.0%. Compared to normal rat. CDAA+vehicle group is the heaviest (1.68, 2.19), CDAA+0.1 mg/kg/day group the intermediate (1.54, 1.21), and CDAA+1.0 mg/kg/day group the lightest (0.82, 1.06), being equivalent to normal (FIG. 18).

### Example 8

Inhibition of Chemical Induced Liver Damage

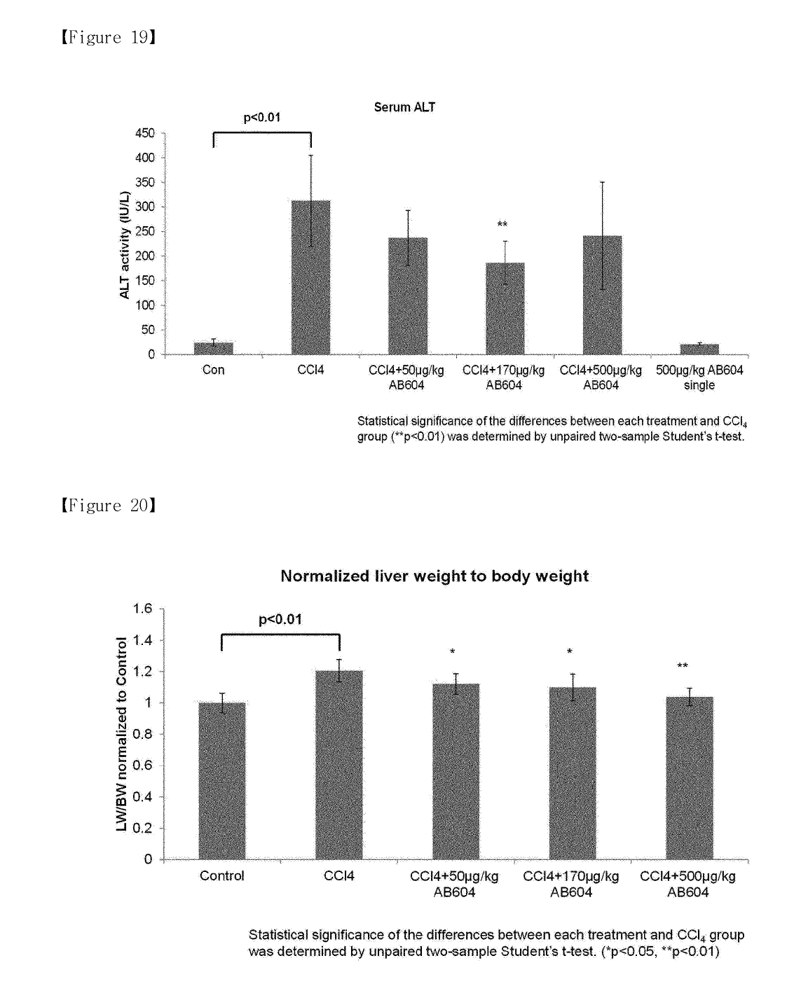

The efficacy of AB604 to inhibit liver fibrosis was tested using CCl₄-induced liver fibrosis model in mice (CCl₄: carbon tetrachloride). CCl₄ works as a toxin to hepatocytes leading to cell death. CCl₄-mediated injuries in liver cause hepatic stellate cells to be activated to myofibroblasts, which proliferate and produce fibrotic material such as αSMA, collagen 1, and fibronectin. C57BL/6 mice received intraperitoneally CCl₄ (0.6 ml/kg) or corn oil twice a week and AB604 was also intraperitoneally injected into CCl₄-treated mice every day during weekdays at 0.05 mg/kg, 0.17 mg/kg, or 0.5 mg/kg. A group that was administered with 0.5 mg/kg AB604 without CCl₄ injection was also included to see toxic effects of AB604, if any. Mice were sacrificed after 2 weeks of treatments and serum ALT levels and relative liver weights were analyzed. Serum ALT level was increased by CCl₄ and reduced by injection of AB604 with statistical significance only at 0.17 mg/kg (FIG. 19). Toxic effects by AB604 were not observed in terms of serum ALT levels. The normalized liver weight to body weight displayed an increase by CCl₄ treatment and this increase was diminished in a dose dependent manner by AB604 treatment (FIG. 20).

1. A composition for treating a liver disease, wherein the composition comprises a chimeric polypeptide having at least 95% sequence identity to an amino acid sequence comprising a first, a second, a third, a fourth, a fifth, and a sixth domains of amino acid residues,
wherein
the first domain of amino acid residues is selected from a group consisting of amino acid residues 1 to X_{1b} of SEQ ID NO:2, amino acid residues 1 to X₁ₐₐ of SEQ ID NO:4, amino acid residues 1 to X_{1ab} of SEQ ID NO:6, amino acid residues 1 to X_{1ac} of SEQ ID NO:8, and amino acid residues 1 to X₁ₐₑ of SEQ ID NO:10;
the second domain of amino acid residues is selected from a group consisting of amino acid residues X_{1b} to X_{2b} of SEQ ID NO:2, amino acid residues X₁ₐₐ to X₂ₐₐ of SEQ ID NO:4, amino acid residues X_{1ab} to X_{2ab} of SEQ ID NO:6, amino acid residues X_{1ac} to X_{2ac} of SEQ ID NO:8, and amino acid residues X₁ₐₑ to X₂ₐₑ of SEQ ID NO: 10;
the third domain of amino acid residues is selected from a group consisting of amino acid residues X_{2b} to X_{3b} of SEQ ID NO:2, amino acid residues X₂ₐₐ to X₃ₐₐ of SEQ ID NO:4, amino acid residues X_{2ab} to X_{3ab} of SEQ ID NO:6, amino acid residues X_{2ac} to X_{3ac} of SEQ ID NO:8, and amino acid residues X₂ₐₑ to X₃ₐₑ of SEQ ID NO: 10;
the fourth domain of amino acid residues is selected from a group consisting of amino acid residues X_{3b} to X_{4b} of SEQ ID NO:2, amino acid residues X₃ₐₐ to X₄ₐₐ of SEQ ID NO:4, amino acid residues X_{3ab} to X_{4ab} of SEQ ID NO:6, amino acid residues X_{3ac} to X_{4ac} of SEQ ID NO:8, and amino acid residues X₃ₐₑ to X₄ₐₑ Of SEQ ID NO:10;
the fifth domain of amino acid residues is selected from a group consisting of amino acid residues X_{4b} to X_{5b} of SEQ ID NO:2, amino acid residues X₄ₐₐ to X₅ₐₐ of SEQ ID NO:4, amino acid residues X_{4ab} to X_{5ab} of SEQ ID NO:6, amino acid residues X_{4ac} to X_{5ac} of SEQ ID NO:8, and amino acid residues X₄ₐₑ to X₅ₐₑ of SEQ ID NO: 10; and
the sixth domain of amino acid residues is selected from a group consisting of amino acid residues X_{5b} to X_{6b} of SEQ ID NO:2, amino acid residues X₅ₐₐ to X₆ₐₐ of SEQ ID NO:4, amino acid residues X_{5ab} to X_{6ab} of SEQ ID NO:6, amino acid residues X_{5ac} to X_{6ac} of SEQ ID NO:8, and amino acid residues X₅ₐₑ to X₆ₐₑ of SEQ ID NO:10;
wherein
X_{1b} is 43, 44, 45, 46, 47, 48, 49, 50, 51, or 52;
X_{2b} is 61, 62, 63, 64, 65, 66, 67, 68, 69, or 70;
X_{3b} is 75, 76, 77, 78, 79, 80, 81, 82, 83, 84, 85, 86, 87, or 88;
X_{4b} is 95, 96, 97, 98, 99, 100, 101, 102, or 103;
X_{5b} is 107, 108, 109, 110, 111, 112, 113, 114, or 115;
X_{6b} is 130, 131, or 132;
X₁ₐₐ is 22, 23, 24, 25, 26, 27, 28, 29, 30, or 31;
X₂ₐₐ is 41, 42, 43, 44, 45, 46, 47, 48, 49, 50, or 51;
X₃ₐₐ is 55, 51, 52, 53, 54, 55, 56, 57, 58, 59, 60, 61, 62, 63, 64, 65, 66, 67, 68, 69, 70, 71, 72, 73, or 74;
X₄ₐₐ is 79, 80, 81, 82, 83, 84, 85, 86, or 87;
X₅ₐₐ is 91, 92, 93, 94, 95, 96, 97, 98, 99, or 100;
X₆ₐₐ is 114, 115, or 116;
X_{1ab} is 22, 23, 24, 25, 26, 27, 28, 29, 30, or 31;
X_{2ab} is 41, 42, 43, 44, 45, 46, 47, 48, 49, 50, or 51;
X_{3ab} is 55, 51, 52, 53, 54, 55, 56, 57, 58, 59, 60, 61, 62, 63, 64, 65, 66, 67, 68, 69, 70, 71, 72, 73, or 74;
X_{4ab} is 78, 79, 80, 81, 82, 83, 84, 85, or 86;
X_{5ab} is 90, 91, 92, 93, 94, 95, 96, 97, 98, or 99;
X_{6ab} is 113, 114, or 115;
X_{1ac} is 22, 23, 24, 25, 26, 27, 28, 29, 30, or 31;
X_{2ac} is 41, 42, 43, 44, 45, 46, 47, 48, 49, 50, or 51;
X_{3ac} is 55, 51, 52, 53, 54, 55, 56, 57, 58, 59, 60, 61, 62, 63, 64, 65, 66, 67, 68, 69, 70, 71, 72, 73, or 74;
X_{4ac} is 79, 80, 81, 82, 83, 84, 85, 86, or 87;
X_{5ac} is 91, 92, 93, 94, 95, 96, 97, 98, 99, or 100;
X_{6ac} is 114, 115, or 116;
X₁ₐₑ is 22, 23, 24, 25, 26, 27, 28, 29, 30, or 31;
X₂ₐₑ is 41, 42, 43, 44, 45, 46, 47, 48, 49, 50, or 51;
X₃ₐₑ is 55, 51, 52, 53, 54, 55, 56, 57, 58, 59, 60, 61, 62, 63, 64, 65, 66, 67, 68, 69, 70, 71, 72, 73, or 74;
X₄ₐₑ is 77, 78, 79, 80, 81, 82, 83, 84, or 85;
X₅ₐₑ is 89, 90, 91, 92, 93, 94, 95, 96, 97, or 98; and
X₆ₐₑ is 112, 113, or 114; and
wherein the chimeric polypeptide is capable of binding to one or more of Transforming Growth Factor-beta (TGF-β) superfamily members; or one or more of TGF-β receptors.

2. The composition of claim 1, wherein said liver disease is any one of liver diseases, disorders, or damages where modulating TGF-beta activity provides a therapeutic benefit.

3. The composition of claim 1, wherein said liver disease is non-alcoholic fatty liver disease, liver fibrosis, or hepatic inflammation.

4. The composition of claim 1, wherein the sequence of said polypeptide is described by an algorithm 1n2n3n4n5n6n,
wherein said 1n, 2n, 3n, 4n, 5n, and 6n represent respectively the first, second, third, fourth, fifth, and sixth domain; and said n is either a or b, and
wherein said a represents an amino acid sequence derived from the sequence of SEQ ID NO:2; and said b represents an amino acid sequence derived from any one selected from the group consisting of SEQ ID NO:4, SEQ ID NO:6, SEQ ID NO:8, and SEQ ID NO:10.

5. The composition of claim 4, wherein the sequence of said polypeptide is described by an algorithm 1b2a3b4b5a6a.

6. The composition of claim 1, wherein
said first domain comprises amino acid residues 1 to 47 of SEQ ID NO:2;
said second domain comprises amino acid residues 28 to 45 of SEQ ID NO:4;
said third domain comprises amino acid residues 66 to 85 of SEQ ID NO:2;
said fourth domain comprises amino acid residues 86 to 99 of SEQ ID NO:2;
said fifth domain comprises amino acid residues 84 to 95 of SEQ ID NO:4; and
said sixth domain comprises amino acid residues 96 to 116 of SEQ ID NO:4.

7. The composition of claim 1, wherein said polypeptide comprises the sequence as set forth in SEQ ID NO:12.

8. The composition of claim 1, wherein said polypeptide has at least 95% sequence identity to the sequence as set forth in SEQ ID NO:12.

9. The composition of claim 1, wherein said chimeric polypeptide has at least 97% sequence identity to the amino acid sequence comprising said first domain, said second domain, said third domain, said fourth domain, said fifth domain, and said sixth domain.

10. The composition of claim 1, wherein the said polypeptide forms a homo-dimer.

11. The composition of claim 1, wherein the said polypeptide forms a hetero-dimer.

12. A method for treating a liver disease comprising administering a therapeutically effective amount of a composition comprising the chimeric polypeptide of claim 1 to a subject in need thereof.

13. The composition of claim 2, wherein the said polypeptide forms a homo-dimer.

14. The composition of claim 2, wherein the said polypeptide forms a hetero-dimer.

15. The composition of claim 3, wherein the said polypeptide forms a homo-dimer.

16. The composition of claim 3, wherein the said polypeptide forms a hetero-dimer.

17. The composition of claim 4, wherein the said polypeptide forms a homo-dimer.

18. The composition of claim 4, wherein the said polypeptide forms a hetero-dimer.

19. The composition of claim 5, wherein the said polypeptide forms a homo-dimer.

20. The composition of claim 5, wherein the said polypeptide forms a hetero-dimer.

21. The composition of claim 6, wherein the said polypeptide forms a homo-dimer.

22. The composition of claim 6, wherein the said polypeptide forms a hetero-dimer.

23. The composition of claim 7, wherein the said polypeptide forms a homo-dimer.

24. The composition of claim 7, wherein the said polypeptide forms a hetero-dimer.

25. The composition of claim 8, wherein the said polypeptide forms a homo-dimer.

26. The composition of claim 8, wherein the said polypeptide forms a hetero-dimer.

27. The composition of claim 9, wherein the said polypeptide forms a homo-dimer.

28. The composition of claim 9, wherein the said polypeptide forms a hetero-dimer.


SEQUENCE LISTING (12 sequences)

SEQ ID NO: 1 - DNA, 396 bp, Homo sapiens
caacagagtcgtaatcgctctacccagtcccaggacgtggcgcgggtctccagtgcttcagattacaacagcagtgaattgaaaacagcctgcaggaagcatgagctgtatgtgagtttccaagacctgggatggcaggactggatcattgcacccaagggctatgctgccaattactgtgatggagaatgctccttcccactcaacgcacacatgaatgcaaccaaccacgcgattgtgcagaccttggttcaccttatgaaccccgagtatgtccccaaaccgtgctgtgcgccaactaagctaaatgccatctcggttctttactttgatgacaactccaatgtcattctgaaaaaatacaggaatatggttgtaagagcttgtggatgccac

SEQ ID NO: 2 - protein, 132 aa, Homo sapiens
QQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 3 - DNA, 348 bp, Homo sapiens
ggcttggagtgtgatggcaaggtcaacatctgctgtaagaaacagttctttgtcagtttcaaggacatcggctggaatgactggatcattgctccctctggctatcatgccaactactgcgagggtgagtgcccgagccatatagcaggcacgtccgggtcctcactgtccttccactcaacagtcatcaaccactaccgcatgcggggccatagcccctttgccaacctcaaatcgtgctgtgtgcccaccaagctgagacccatgtccatgttgtactatgatgatggtcaaaacatcatcaaaaaggacattcagaacatgatcgtggaggagtgtgggtgctca

SEQ ID NO: 4 - protein, 116 aa, Homo sapiens [sequence omitted]

SEQ ID NO: 5 - DNA, 345 bp, Homo sapiens
ggcctggagtgcgatggccggaccaacctctgttgcaggcaacagttcttcattgacttccgcctcatcggctggaacgactggatcatagcacccaccggctactacggcaactactgtgagggcagctgcccagcctacctggcaggggtccccggctctgcctcctccttccacacggctgtggtgaaccagtaccgcatgcggggtctgaaccccggcacggtgaactcctgctgcattcccaccaagctgagcaccatgtccatgctgtacttcgatgatgagtacaacatcgtcaagcgggacgtgcccaacatgattgtggaggagtgcggctgcgcc

SEQ ID NO: 6 - protein, 115 aa, Homo sapiens
GLECDGRTNLCCRQQFFIDFRLIGWNDWIIAPTGYYGNYCEGSCPAYLAGVPGSASSFHTAVVNQYRMRGLNPGTVNSCCIPTKLSTMSMLYFDDEYNIVKRDVPNMIVEECGCA

SEQ ID NO: 7 - DNA, 348 bp, Homo sapiens
ggcatcgactgccaaggagggtccaggatgtgctgtcgacaagagttttttgtggacttccgtgagattggctggcacgactggatcatccagcctgagggctacgccatgaacttctgcatagggcagtgcccactacacatagcaggcatgcctggtattgctgcctcctttcacactgcagtgctcaatcttctcaaggccaacacagctgcaggcaccactggagggggctcatgctgtgtacccacggcccggcgccccctgtctctgctctattatgacagggacagcaacattgtcaagactgacatacctgacatggtagtagaggcctgtgggtgcagt

SEQ ID NO: 8 - protein, 116 aa, Homo sapiens
GIDCQGGSRMCCRQEFFVDFREIGWHDWIIQPEGYAMNFCIGQCPLHIAGMPGIAASFHTAVLNLLKANTAAGTTGGGSCCVPTARRPLSLLYYDRDSNIVKTDIPDMVVEACGCS

SEQ ID NO: 9 - DNA, 342 bp, Homo sapiens
acccccacctgtgagcctgcgacccccttatgttgcaggcgagaccattacgtagacttccaggaactgggatggcgggactggatactgcagcccgaggggtaccagctgaattactgcagtgggcagtgccctccccacctggctggcagcccaggcattgctgcctctttccattctgccgtcttcagcctcctcaaagccaacaatccttggcctgccagtacctcctgttgtgtccctactgcccgaaggcccctctctctcctctacctggatcataatggcaatgtggtcaagacggatgtgccagatatggtggtggaggcctgtggctgcagc

SEQ ID NO: 10 - protein, 114 aa, Homo sapiens
TPTCEPATPLCCRRDHYVDFQELGWRDWILQPEGYQLNYCSGQCPPHLAGSPGIAASFHSAVFSLLKANNPWPASTSCCVPTARRPLSLLYLDHNGNVVKTDVPDMVVEACGCS

SEQ ID NO: 11 - DNA, 396 bp, Artificial Sequence
  Synthetic construct (AB604 DNA)
caacagagtcgtaatcgctctacccagtcccaggacgtggcgcgggtctccagtgcttcagattacaacagcagtgaattgaaaacagcctgcaggaagcatgagctgtatgtgagtttccaagacctgggatggcaggactggattatcgcgccgagtggctatcacgccaactactgcgagggtgaatgtccgttcccactcaacgcacacatgaatgcaaccaaccacgcgattgtgcagaccttggttcaccttatgaaccccgagtatgtccccaaaccgtgctgtgcgccaaccaaactgcgtccgatgtccatgctgtattacgatgacggccagaacatcatcaaaaaagatatccaaaacatgatcgtcgaagaatgcggttgtagc

SEQ ID NO: 12 - protein, 132 aa, Artificial Sequence
  Synthetic construct (AB604 Polypeptide)
QQSRNRSTQSQDVARVSSASDYNSSELKTACRKHELYVSFQDLGWQDWIIAPSGYHANYCEGECPFPLNAHMNATNHAIVQTLVHLMNPEYVPKPCCAPTKLRPMSMLYYDDGQNIIKKDIQNMIVEECGCS